(19) United States
(12) Patent Application Publication
(10) Pub. No.: US 2002/0122807 A1
(43) Pub. Date: Sep. 5, 2002

(54) Antigen binding fragments, designated 4B5, that specifically detect cancer cells, nucleotides encoding the fragments, and use thereof for the prophylaxis and detection of cancers

(76) Inventors: Michael D. Dan, (US); Mansoor Saleh, (US)

Correspondence Address: MORRISON & FOERSTER LLP, 755 PAGE MILL RD, PALO ALTO, CA, 94304-1018, US

(21) Appl. No.: 09/747,669

(22) Filed: Dec. 21, 2000

Related U.S. Application Data
(63) Continuation of application No. 09/111,286, filed on Jul. 7, 1998, now abandoned.

(51) Int. Cl. A61K 39/395; C12Q 1/68; C07H 21/04

(52) U.S. Cl. 424/181.1; 435/6; 536/23.5

(57) ABSTRACT

The present invention relates to monoclonal antibody 4B5 and antigen binding fragments that specifically bind to the antibodies specific for GD2. Also disclosed are polynucleotide and polypeptide derivatives based on 4B5, including single chain V region molecules and fusion proteins, and various pharmaceutical compositions. When administered to an individual, the 4B5 antibody is effective in diagnosing, and/or treating neoplasias. The invention further provides methods for treating a neoplastic disease, particularly melanoma, neuroblastoma, glioma, soft tissue sarcoma, and lung carcinomas. Patients who are in remission as a result of traditional modes of cancer therapy can be treated with a composition of this invention in hopes of reducing the risk of recurrence. Patients can also be treated concurrently with the antibodies and traditional anti-neoplastic agents.

### TECHNICAL FIELD

This invention relates to polynucleotides that encode anti-idiotypic antibodies that mimic the GD2 antigen. The recombinant human monoclonal antibody (Mab) and antigenic fragments thereof are termed “4B5.” The invention encompasses a wide variety of antibodies, and functional derivatives thereof that retain the immunologic specificity of 4B5 and are termed herein “4B5.” The polynucleotides encoding 4B5 and polypeptides encoded thereby and recombinant molecules containing these polynucleotides are also encompassed by the invention. Methods of use including therapeutic and diagnostic of the 4B5 antibodies are also included in the invention.

### BACKGROUND ART

In spite of numerous advances in medical research, cancer remains the second leading cause of death in the United States. In the industrialized nations, roughly one in five persons will die of cancer. Traditional modes of clinical care, such as surgical resection, radiotherapy and chemotherapy, have a significant failure rate, especially for solid tumors. Failure occurs either because the initial tumor is unresponsive, or because of recurrence due to regrowth at the original site and/or metastases. Even in cancers such as breast cancer where the mortality rate has decreased, successful intervention relies on early detection of the cancerous cells. The etiology, diagnosis and ablation of cancer remain a central focus for medical research and development.

Neoplasia resulting in benign tumors can usually be completely cured by removing the mass surgically. If a tumor becomes malignant, as manifested by invasion of surrounding tissue, it becomes much more difficult to eradicate. Once a malignant tumor metastasizes, it is much less likely to be eradicated.

The three major cancers, in terms of morbidity and mortality, are colon, breast and lung. New surgical procedures offer an increased survival rate for colon cancer. Improved screening methods increase the detection of breast cancer, allowing earlier, less aggressive therapy. Numerous studies have shown that early detection increases survival and treatment options. Lung cancer remains largely refractory to treatment.

Excluding basal cell carcinoma, there are over one million new cases of cancer per year in the United States alone, and cancer accounts for over one half million deaths per year in this country. In the world as a whole, the five most common cancers are those of lung, stomach, breast, colon/rectum, and uterine cervix, and the total number of new cases per year is over 6 million. Only about half the number of people who develop cancer die of it.

Melanoma is one of the human diseases for which there is an acute need of new therapeutic modalities. It is a particularly aggressive form of skin cancer, and occurs in increased frequency in individuals with regular unguarded sun exposure. In the early disease phases, melanoma is characterized by proliferation at the dermal-epidermal junction, which soon invades adjacent tissue and metastasizes widely. Once it has metastasized, it is often impossible to extirpate and is consequently fatal. Worldwide, 70,000 patients are diagnosed with melanoma and it is responsible for 25,000 reported deaths each year. The American Cancer Society projects that by the year 2000, 1 out of every 75 Americans will be diagnosed with melanoma.

Neuroblastoma is a highly malignant tumor occurring during infancy and early childhood. Except for Wilm's tumor, it is the most common retroperitoneal tumor in children. This tumor metastasizes early, with widespread involvement of lymph nodes, liver, bone, lung, and marrow. While the primary tumor is resolvable by resection, the recurrence rate is high.

An estimated 178,100 new cases of lung cancer will be diagnosed in 1997, accounting for 13% of cancer diagnoses. An estimated 160,400 deaths due to lung cancer will occur in 1997 accounting for 29% of all cancer deaths. The one year survival rates for lung cancer have increased from 32% in 1973 to 41% in 1993, largely due to improvements in surgical techniques. The 5 year survival rate for all stages combined is only 14%. The survival rate is 48% for cases detected when the disease is still localized, but only 15% of lung cancers are discovered that early.

Small cell lung cancer is the most malignant and fastest growing form of lung cancer and accounts for 20-25% of new cases of lung cancer. 60,000 cases will be diagnosed in the U.S. in 1996. The primary tumor is generally responsive to chemotherapy, but is followed by wide-spread metastasis. The median survival time at diagnosis is approximately 1 year, with a 5 year survival rate of 5-10%.

Breast cancer is one of the most common cancers and is the third leading cause of death from cancers in the United States with an annual incidence of about 180,200 new cases among women in the United States during 1997. About 1,400 new cases of breast cancer will be diagnosed in men in 1997. In industrialized nations, approximately one in eight women can expect to develop breast cancer. The overall mortality rate for breast cancer has remained unchanged since 1930. It has increased an average of 0.2% per year, but decreased in women under 65 years of age by an average of 0.3% per year. Preliminary data suggest that breast cancer mortality may be beginning to decrease, probably as a result of increased diagnoses of localized cancer and carcinoma in situ. See e.g., Marchant (1994) Contemporary Management of Breast Disease II: Breast Cancer, in: Obstetrics and Gynecology Clinics of North America 21:555-560; and Colditz (1993) Cancer Suppl. 71:1480-1489. An estimated 44,190 deaths (43,900 women, 290 men) in 1997 will occur due to breast cancer. In women, it is the second major cause of cancer death after lung cancer. The five-year survival rate for localized breast cancer has increased from 72% in the 1940s to 97% today. If the cancer has spread regionally, however, the rate is 76%, and for women with distant metastases the rate is 20%. Survival after a diagnosis of breast cancer continues to decline beyond five years. Sixty-five percent of women diagnosed with breast cancer survive 10 years and 56% survive 15 years.

Non-Hodgkin's B cell lymphomas are cancers of the immune system that are expected to afflict approximately 225,000 patients in the United States in 1996. These cancers are diverse with respect to prognosis and treatment, and are generally classified into one of three grades. The median survival of the lowest grade is 6.6 years and the higher grade cancers have much lower life expectancy. Virtually all non-Hodgkin's B cell lymphomas are incurable. New diagnoses of non-Hodgkins lymphomas have increased approximately 7% annually over the past decade, with 52,700 new diagnoses estimated for 1996. The increase is due in part to the increasing prevalence of lymphomas in the AIDS patient population.

Colon and rectal cancer will account for an estimated 131,200 cases in 1997, including 94,100 of colon cancer and 37,100 of rectal cancer. Colorectal cancers account for about 9% of new cancer diagnoses. An estimated 54,900 deaths due to colorectal cancer will occur in 1997, accounting for about 10% of cancer deaths. Mortality rates for colorectal cancer have fallen 32% for women and 14% for men during the past 20 years, reflecting decreasing incidence rates and increasing survival rates. However, the mortality rate in African American men continues to rise. The 1 and 5 year relative survival rates for patients with colon and rectal cancer are 82% and 61%, respectively. When colorectal cancers are detected in an early, localized stage, the 5 year survival rate is 91%; however, only 37% of colorectal cancers are discovered at that stage. After the cancer has spread regionally to involve adjacent organs or lymph nodes, the rate drops to 63%. Survival rates for persons with distant metastases is 7%. Survival continues to decline beyond 5 years, and 50% survive 10 years.

In spite of the difficulties, effective cures using anticancer drugs (alone or in combination with other treatments) have been devised for some formerly highly lethal cancers. Most notable among these are Hodgkin's lymphoma, testicular cancer, choriocarcinoma, and some leukemias and other cancers of childhood. For several of the more common cancers, early diagnosis, appropriate surgery or local radiotherapy enables a large proportion of patients to recover.

Current methods of cancer treatment are relatively non-selective. Surgery removes the diseased tissue, radiotherapy shrinks solid tumors and chemotherapy kills rapidly dividing cells. Chemotherapy, in particular, results in numerous side effects, in some cases so severe to preclude the use of potentially effective drugs. Moreover, cancers often develop resistance to chemotherapeutic drugs.

Numerous efforts are being made to enhance the specificity of cancer therapy. For review, see Kohn and Liotta (1995) Cancer Res. 55:1856-1862. In particular, identification of cell surface antigens expressed exclusively or preferentially on certain tumors allows the formulation of more selective treatment strategies. Antibodies directed to these antigens have been used in immunotherapy of several types of cancer.

The basic immunoglobulin (Ig) structural unit in vertebrate systems is composed of two identical light (“L”) polypeptide chains (approximately 23 kDa), and two identical heavy (“H”) chains (approximately 53 to 70 kDa). The four chains are joined by disulfide bonds in a “Y” configuration. At the base of the Y, the two H chains are bound by covalent disulfide linkages.

FIG. 1 shows a schematic of an antibody structure. The L and H chains are each composed of a variable (V) region at the N-terminus, and a constant (C) region at the C-terminus. In the L chain, the V region (termed “V_{L}J_{L}”) is composed of a V (V_{L}) region connected through the joining (J_{L}) region to the C region (C_{L}). In the H chain, the V region (V_{H}D_{H}J_{H}) is composed of a variable (V_{H}) region linked through a combination of the diversity (D_{H}) region and the joining (J_{H}) region to the C region (C_{H}). The V_{L}J_{L} and V_{H}D_{H}J_{H} regions of the L and H chains, respectively, are associated at the tips of the Y to form the antigen binding portion and determine antigen binding specificity.

The (C_{H}) region defines the isotype, i.e., the class or subclass of antibody. Antibodies of different isotypes differ significantly in their effector functions, such as the ability to activate complement, bind to specific receptors (e.g., Fc receptors) present on a wide variety of cell types, cross mucosal and placental barriers, and form polymers of the basic four-chain IgG molecule.

Antibodies are categorized into “classes” according to the C_{H} type utilized in the immunoglobulin molecule (IgM, IgG, IgD, IgE, or IgA). There are at least five types of C_{H} genes (Cμ, Cγ, Cδ, Cε, and Cα), and some species have multiple C_{H} subtypes (e.g., Cγ₁, Cγ₂, Cγ₃, and Cγ₄, in humans). There are a total of nine C_{H} genes in the haploid genome of humans, eight in mouse and rat, and several fewer in many other species. In contrast, there are normally only two types of L chain C regions (C_{L}), kappa (τ) and lambda (λ), and only one of these C regions is present in a single L chain protein (i.e., there is only one possible L chain C region for every V_{L}J_{L} produced). Each H chain class can be associated with either of the L chain classes (e.g., a C_{H}γ region can be present in the same antibody as either a τ or λL chain), although the C regions of the H and L chains within a particular class do not vary with antigen specificity (e.g., an IgG antibody always has a CγH chain C region regardless of the antigen specificity).

Each of the V, D, J, and C regions of the H and L chains are encoded by distinct genomic sequences. Antibody diversity is generated by recombination between the different V_{H}, D_{H}, and J_{H}, gene segments in the H chain, and V_{L} and J_{L} gene segments in the L chain. The recombination of the different V_{H}, D_{H}, and J_{H} genes is accomplished by DNA recombination during B cell differentiation Briefly, the H chain sequence recombines first to generate a D_{H}J_{H} complex, and then a second recombinatorial event produces a V_{H}D_{H}J_{H} complex. A functional H chain is produced upon transcription followed by splicing of the RNA transcript. Production of a functional H chain triggers recombination in the L chain sequences to produce a rearranged V_{L}J_{L} region which in turn forms a functional V_{L}J_{L}C_{L} region, i.e., the functional L chain.

The value and potential of antibodies as diagnostic and therapeutic reagents has been long-recognized in the art. Unfortunately, the field has been hampered by the slow, tedious processes required to produce large quantities of an antibody of a desired specificity. The classical cell fusion techniques allowed for efficient production of Mabs by fusing the B cell producing the antibody with an immortalized cell line. The resulting cell line is a hybridoma cell line.

Antibodies and functional derivatives thereof have been used in a variety of clinical settings. For instance, digoxin-specific Fab antibody fragments were used to treat life-threatening digitalis intoxication. Antibodies are becoming more routinely useful in diagnostic techniques such as radioimmune diagnosis of colon cancers. Koda et al. (1995) Am. J Gastroenterol. 90:1644. A number of uses of Mabs, previously thought to be untenable, have recently been put into practice. For instance, see Hall (1995) Science 279:915-916.

A number of autoantibodies (antibodies that recognize and bind to self antigens) are found in humans. Many of these are associated with particular diseases such as rheumatoid arthritis, systemic lupus erythematosus, myasthenia gravis, primary biliary cirrhosis, polymyositis, systemic vasculitis, idiopathic necrotizing and crescentic glomerulonephritis and amyotrophic lateral sclerosis. For review, see Shattner (1986/1987) Immunol. Lett. 14:143-153. Other autoantibodies are naturally-occurring. Lutz and Wipp (1982) J. Immunol. 128:1965; and Guilbert et al. (1982) J. Immunol. 128:2779-2787. Recently, human autoantibodies to specific cancer antigens have been detected and, in some cases, are being produced by hybridoma technology. These antibodies have also been produced by active immunization. U.S. Pat. No. 5,474,755. Originally, the human B cells were immortalized using Epstein-Barr Virus or mouse myelomas. For review, see Buck et al. (1984) “Monoclonal Antibodies” NY, Plenum Press. More recent techniques have allowed immortalization without the use of this potentially harmful virus. See, e.g., U.S. Pat. No. 4,618,477; and Glassy (1987) Cancer Res. 47:5181-5188. In most instances, the antibodies are specific for one, or in some instances, a few, cancer types. For instance, a Mab has been described that specifically recognizes glioma cells but no other tumor or normal cells. These antibodies were used to image the glioma in the patient's brain. Fischer et al. (1991) Immunobiol. Prot. Pep. VI (M. Atassi, ed.) Plenum Press, NY. pp. 263-270. No antibody has been described that is capable of recognizing a wide range of tumors while failing to recognize, or only poorly recognize, normal, non-cancerous cells.

Recombinant genetic techniques have allowed cloning and expression of antibodies, functional fragments thereof and the antigens recognized. These engineered antibodies provide novel methods of production and treatment modalities. For instance, functional immunoglobulin fragments have been expressed in bacteria and transgenic tobacco seeds and plants. Skerra (1993) Curr. Opin. Immunol. 5:256-262; Fiedler and Conrad (1995) Bio/Technology 13:1090-1093; Zhang et al. (1993) Cancer Res. 55:3384-3591; Ma et al. (1995) Science 268:916; and, for a review of synthetic antibodies, see Barbas (1995) Nature Med. 1:836-839.

Several human Mabs against tumor associated antigens have been produced and characterized. The tumor associated antigens recognized by human Mabs include cell surface, cytoplasmic and nuclear antigens. Yoshikawa et al. (1989) Jpn. J. Cancer Res. (Gann) 80:546-553; Yamaguchi et al. (1987) Proc. Natl. Acad. Sci. USA 84:2416-2420; Haspel et al. (1985) Cancer Res. 45:3951-3961; Cote et al. (1986) Proc. Natl. Acad. Sci. USA 83:2959-2963; Glassy (1987) Cancer Res. 47:5181-5188; Borup-Christensen et al. (1987) Cancer Detect. Prevent. Suppl. 1:207-215; Haspel et al. (1985) Cancer Res. 45:3951-3961; Kan-Mitchell et al. (1989) Cancer Res. 49:4536-4541; Yoshikawa et al. (1986) Jpn. J. Cancer Res. 77:1122-1133; and McKnight et al. (1990) Human Antibod. Hybridomas 1: 125-129. Dan et al. (1992) J. Neurosurg. 76: 660-669.

Human Mabs have been used in cancer imaging, diagnosis and therapy. Olsson (1985) J. Nat. Cancer Inst. 75:397-404; Larrick and Bourla (1986) J. Biol. Resp. Mod. 5:379-393; McCabe et al. (1988) Cancer Res. 48:4348-4353; Research News (1993) Science 262:841; Ditzel et al. (1994) Cancer 73:858-863; and Alonso (1991) Am. J. Clin. Oncol. 4:463471. A recombinant single chain bispecific antibody has been reported that has high tumor cell toxicity. This molecule recognizes both the CD3 antigen of human T cells and EpCAM, which is associated with disseminated tumor cells in patients with minimal residual colorectal cancer. Mack et al. (1995) Proc. Natl. Acad. Sci. USA 92:7021-7025.

Immunobiologists have learned that a poor antigen (in terms of eliciting an immune response) can be turned into a strong antigen by changing the molecular environment. Changes of hapten carrier allow T cell helper cells to become active, making the overall immune response stronger. Thus, changing the carrier can also turn a tolerogenic antigen into an effective antigen. McBridge et al. (1986) Br. J. Cancer 53:707. Often the immunological status of a cancer patient is suppressed so that the patient is only able to respond to certain T-dependent antigens and not to other antigen forms. From these considerations, it would make sense to introduce molecular changes into the tumor associated antigens before using them as vaccines. Unfortunately, this is impossible to accomplish for most tumor antigens, because they are not well defined and are very hard to purify.

The network hypothesis of Lindemann ((1973) Ann. Immunol. 124:171-184) and Jerne ((1974) Ann. Immunol. 125:373-389) offers an elegant approach to transform epitope structures into idiotypic determinants expressed on the surface of antibodies. According to the network concept, immunization with a given tumor-associated antigen will generate production of antibodies against this tumor-associated antigen, termed Ab1; this Ab1 is then used to generate a series of anti-idiotype antibodies against the Ab1, termed Ab2. Some of these Ab2 molecules can effectively mimic the three-dimensional structure of the tumor-associated antigen identified by the Ab1. These particular anti-idiotypes called Ab2β fit into the paratopes of Ab1, and express the internal image of the tumor-associated antigen. The Ab2β can induce specific immune responses similar to those induced by the original tumor-associated antigen and can, therefore, be used as surrogate tumor-associated antigens. Immunization with Ab2β can lead to the generation of anti-anti-idiotype antibodies (Ab3) that recognize the corresponding original tumor-associated antigen identified by Ab1. Because of this Ab1-like reactivity, the Ab3 is also called Ab1′ to indicate that it might differ in its other idiotopes from Ab1.

A potentially promising approach to cancer treatment is immunotherapy employing anti-idiotype antibodies. In this form of therapy, an antibody mimicking an epitope of a tumor-associated protein is administered in an effort to stimulate the patient's immune system against the tumor, via the tumor-associated protein. WO 91/11465 describes methods of stimulating an immune response in a human against malignant cells or an infectious agent using primate anti-idiotype antibodies. However, not all anti-idiotype antibodies can be used in therapeutic regimens against tumors. First, only a fraction of antibodies raised against an Ab1 are limited in their reactivity to the paratope of Ab1 (i.e., are non-reactive against features shared with other potential antibodies in the host). Second, anti-idiotype antibodies are not necessarily immunogenic. Third, even if an anti-idiotype elicits an immune response, only a fraction of these immunogenic anti-idiotypes elicit an immune response against the tumor antigen and not against other antigens with less specificity. Moreover, since different cancers have widely varying molecular and clinical characteristics, it has been suggested that anti-idiotype therapy should be evaluated on a case by case basis, in terms of tumor origin and antigens express.

Anti-Id monoclonal antibodies structurally resembling tumor-associated antigens have been used as antigen substitutes in cancer patients. Herlyn et al. (1987) Proc. Natl. Acad. Sci. USA 84:8055-8059; Mittleman et al. (1992) Proc. Natl. Acad. Sci. USA 89:466-470; Chatterjee et al. (1993) Ann. N.Y. Acad. Sci. 690:376-377. It has been proposed that the anti-Id provides a partial analog of the tumor-associated antigen in an immunogenic context.

Several murine monoclonal anti-GD2 antibodies were reported to suppress the growth of tumors of neuroectodermal origin in athymic (nu/nu) mice or cause remission in patients with metastatic melanoma. Several antibodies specific for GD2 have been described and deposited with the ATCC. These are HB-9326, described in U.S. Pat. No. 4,849,509; HB-9325, described in U.S. Pat. No. 4,849,509; and HB-8568, described in U.S. Pat. No. 4,675,287. Anti-idiotypic monoclonal antibodies providing an internal anti-idiotypic determinant on an antibody to GD2 have also been described. U.S. Pat. No. 5,612,030; and Saleh et al. (1993) J. Immunol. 151:3390-3398. The anti-idiotypic antibodies are described for use in treatment of melanoma and small cell carcinoma. A human-mouse chimeric anti-GD2 antibody caused remission in patients with metastatic neuroblastoma. The mechanism of action of the antibodies is thought to involve antibody dependent cellular cytotoxicity (ADCC) or complement-mediated cytotoxicity (CMC). Clinical responses have been obtained by treating melanoma with Mabs against GM2, GD2 and GD3. Cheresh et al. (1985) Proc. Natl. Acad. Sci. USA 82:5155-5159. Active immunization with a ganglioside vaccine comprising GM2 produced anti-GM2 antibodies in 50/58 patients, who survived longer on average than patients without detectable anti-GM2 antibody.

Mabs to GD2 have been found to react specifically with small cell lung carcinoma. Cheresh et al. (1986) Cancer Res. 46:5112-5118. Human Mabs specific for other cancers including lung, melanoma, stomach, squamous cell carcinoma, cervical carcinoma, and mammary carcinoma have also been produced. Murakami (1985) in Vitro Cell. Dev. Biol. 21:593; Schadendorf (1989) J. Immunol. 142:1621-1625; Yoshikawa et al. (1986) Jpn. J. Cancer Res. 77:1122-1133; Pickering and Misra (1984) Clin. Immunol. Immunopathol. 32:253-260; Hagiwara and Sato (1983) Mol. Biol. Med. 1:245-252; and Schlom et al. (1980) Proc. Natl. Acad. Sci. USA 77:6841-6845. Human anti-cancer Mabs and the antigens they recognize have also been suggested for use in vaccines. See, e.g. Finn et al. (1995) Immunol. Rev. 145:61-89. A human Mab to malignant brain tumors was used in a phase I clinical trial without adverse side effects. Matsumoto et al. (1994) The Clinical Report 28:118-126. Phase II trial results have been reported on combined treatment with murine Mab and colony stimulating factor in metastatic gastrointestinal cancer. Saleh et al. (1995) Cancer Res. 55:4339-4346. A single chain immunotoxin has also been found to cure carcinomatous meningitis in a rat model. Pastan et al. (1995) Proc. Natl. Acad. Sci. USA 92:2765-2769. Human Mabs that specifically recognize ovarian cancer cells have been shown to effectively image this cancer. Chaudhuri et al. (1994) Cancer 73:1098-1104.

If there were a simple and reliable strategy for providing immune reactivity against an antigen common to these cancers rather than cancer-specific immunity, the clinical prospects of cancers in general would improve. All references cited herein are hereby incorporated by reference in their entirety.

### DISCLOSURE OF THE INVENTION

This invention encompasses compositions containing antigen binding fragments of an antibody where the antibody specifically recognizes the antigen recognized by an antibody comprising a H chain having the amino acid sequence of SEQ ID NO: 2 and a L chain having the amino acid sequence of SEQ ID NO: 4. Preferably, the antibody is recombinant 4B5. The invention further encompasses antibodies comprising the H and L chain of 4B5 (SEQ ID NOS: 2 and 4, respectively). 4B5 specifically recognizes cancer cells from a wide variety of cancers but does not recognize normal, non-cancerous cells. By “does not recognize” is meant that noncancer cells are either not specifically bound to by 4B5 or are only poorly recognized by the antibody. These antigen binding fragments include, but are not limited to, whole native antibodies, exemplified by the recombinant 4B5 described herein; bispecific antibodies; chimeric antibodies; Fab, Fab′, single chain V region fragments (scFv) and fusion polypeptides.

The invention further encompasses 4B5 antibody fusion molecules comprising a polypeptide region with an antigenic, therapeutic, toxic or labeling molecule attached to the H chain C region, a single-chain V_{H}—V_{L} or V_{L}—V_{H} V region, and polynucleotides encoding such polypeptides.

Also embodied in the invention is a polynucleotide comprising a sequence encoding a polypeptide with the immunologic specificity of 4B5, wherein the encoded-polypeptide comprises at least 5 consecutive amino acids from a V region of 4B5. The V region may be from either the 4B5 L chain or H chain. The 5 consecutive amino acids preferably play a role in 4B5 immunologic reactivity, and may be from a CDR.

The invention also encompasses isolated polynucleotides of at least 20 consecutive nucleotides capable of forming a stable duplex with the 4B5 L or H chain encoding sequences, but not with sequences for other previously described immunoglobulin molecules. Any of these polynucleotides can be in the form of cloning vectors, expression vectors, or transfected into host cells.

Another embodiment of this invention are polypeptides having the immunologic specificity of 4B5, wherein the polypeptide comprises at least 5 consecutive amino acids from a V region of an 4B5 antibody. The V region may be from a L chain or H chain. The 5 consecutive amino acids preferably play a role in immunologic specificity, and may be from a CDR (Complementarity Determining Region of an antibody). Intact recombinant 4B5, functionally active fragments of 4B5, fusion proteins, chimeric antibodies, multiple antigen proteins, and other polypeptide derivatives of 4B5 antibodies are included. Of special interest are single-chain V regions and fusion proteins.

The compounds and compositions of this invention can be used inter alia for detecting or treating a cancer; including therapy of such cancer, and prophylactic care, particularly for decreasing the risk of recurrence.

The invention further embodies cells and cell lines producing 4B5.

A further embodiment of this invention comprises prophylactic treatment of a cancer patient with at least one 4135 antigen binding fragment. Preferably, 4B5 is fused to a therapeutic molecule to effect delivery of the therapeutic molecule to the cancer cell. The individual may have a clinically detectable tumor, or the tumor may have been previously treated and rendered undetectable. The method can be for palliating the disease, or for reducing the risk of recurrence.

A further embodiment of the invention is a kit for detection or quantitation of the antibodies specific for GD2 in a sample, comprising 4B5 in suitable packaging.

### BRIEF DESCRIPTION OF THE DRAWINGS

FIG. 1 depicts a schematic of the general antibody structure. FIG. 2 depicts the purification of recombinant 4B5 on protein G Sepharose column. The 4B5 antibodies produced by the hybridoma cells are shown in lane 1 and 2. Recombinant 4B5 antibodies are shown in lane 3 and 4. Lane 1 and 3 contain 3μg of protein and lane 2 and 4 contain 6μg of protein.

FIG. 3 depicts the determination of the antigenic similarity between recombinant 4B5 and hybridoma 4B5 by ELISA analysis. Dark bars represent binding of the 4B5 antibodies or control human IgG to antigen 14G2A. Light bars represent non-specific binding of the 4B5 antibodies or control human IgG to mouse IgG.

FIG. 4 depicts the binding specificity of recombinant 4B5 to antigen 14G2A by Western blot analysis.

FIG. 5 depicts the plasmid expression vector pNB2-4B5.

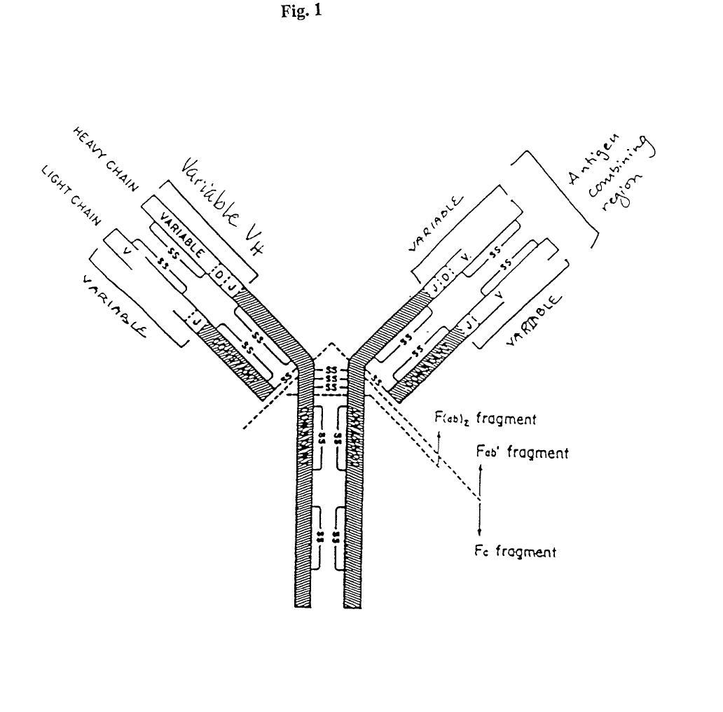

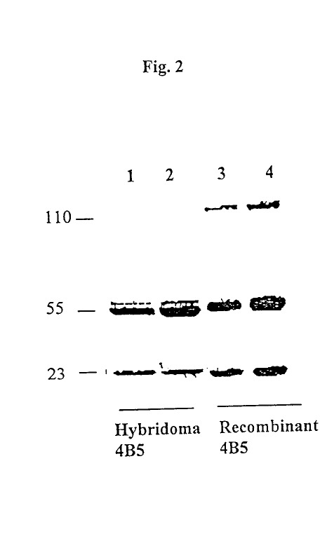

### BEST MODE FOR CARRYING OUT THE INVENTION

This invention encompasses antigen binding fragments exemplified by the recombinant antibody described herein. The exemplary antibody is designated 4B5 and is encoded by SEQ ID NOS: 1 and 4 (SEQ ID NOS: 2 and 5 being the complementary strands of 1 and 4, respectively) and recognizes antibodies specific for GD2 antibodies. Antibodies specific for GD2 recognize various cancers including, but not limited to, glioblastoma, neuroblastoma, malignant and/or metastatic melanoma, breast adenocarcinoma, lung adenocarcinoma, small cell lung carcinoma, colon adenocarcinoma and prostate adenocarcinoma.

The invention further encompasses 4B5 derivatives with immunologic specificity for antibodies specific for GD2. These derivatives comprise regions of the polypeptide sequence comprising part of the 4B5 VDJ junction. Also encompassed are regions spanning at least one, preferably 2, and more preferably 3 or more of the 4B5 CDR amino acid sequences.

Certain compounds, compositions and methods described in this application relate generally to 4B5 and derivatives thereof which are routinely generated by classical techniques of immunochemistry. This includes 4B5 which has been coupled to another compound by chemical conjugation, or by mixing with an excipient or an adjuvant. The term antigen binding fragment includes any peptide that is recognized specifically by antibodies specific for GD2. Typically, these derivatives include such immunoglobulin fragments as Fab, F(ab′)₂, Fab′, scFv (both monomers and polymeric forms) and isolated H and L chains. An antigen binding fragment retains the antigenic specificity of 4B5, although avidity and/or affinity may be altered.

The antigen binding fragments (also termed “derivatives” herein) are typically generated by genetic engineering, although they may alternatively be obtained by other methods and combinations of methods. This classification includes, but is not limited to, engineered peptide fragments and fusion peptides. Preferred compounds include polypeptide fragments of the 4B5 CDRs, antibody fusion proteins comprising cytokine effector components, antibody fusion proteins comprising adjuvants or drugs, and single-chain V region proteins.

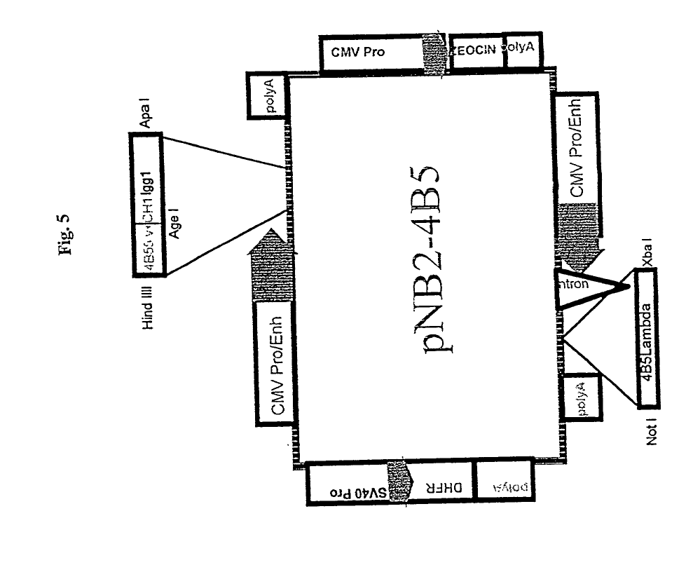

The invention further comprises polynucleotides encoding the 4B5 antibody V regions and derivatives thereof. These include isolated polynucleotide fragments both coding and complimentary strands, recombinant polynucleotides, and therapeutic plasmids and vectors, such as vaccinia vectors, comprising the polynucleotides. These polynucleotides are exemplified by SEQ ID NOS: 1, 2, 4 and 5 and plasmid pNB2-4B5 (FIG. 5).

As 4B5 has been shown to mimic GD2, it is particularly useful in generating a host immune response to cancer. Pharmaceutical compositions and treatment modalities of this invention are suitable for eliciting an immune response against neoplasia. Suitable carcinomas include any known in the field of oncology, including, but not limited to, astrocytoma, oligodendroglioma, ependymoma, medulloblastoma, primitive neural ectodermal tumor (PNET), pancreatic ductal adenocarcinoma, small and large cell lung adenocarcinomas, squamous cell carcinoma, bronchoalveolarcarcinoma, epithelial adenocarcinoma, and liver metastases thereof, hepatoma, cholangiocarcinoma, breast tumors such as ductal and lobular adenocarcinoma, squamous and adenocarcinomas of the uterine cervix, uterine and ovarian epithelial carcinomas, prostatic adenocarcinomas, transitional squamous cell carcinoma of the bladder, B and T cell lymphomas (nodular and diffuse) plasmacytoma, acute and chronic leukemias, malignant melanoma, soft tissue sarcomas and leiomyosarcomas.

The subjects may have an advanced form of disease, in which case the treatment objective may include mitigation or reversal of disease progression, and amelioration of side effects. The subjects may have had a history of the condition, for which they have already been treated, in which case the objective will typically include a decrease or delay in the risk of recurrence.

Immunologic activity” of 4B5 refers to the ability to specifically bind antibodies specific for GD2. Such binding may or may not elicit an immune response. A specific immune response may comprise antibody, B cells, T cells, and any combination thereof, and effector fimctions resulting therefrom. Included are the antibody-mediated functions ADCC and complement-mediated cytolysis (CDC). The T cell response includes T helper cell function, cytotoxic T cell function, inflammation/inducer T cell function, and T cell mediated suppression. A compound able to elicit a specific immune response according to any of these criteria is referred to as “immunogenic.”

4B5 “activity” or “function” refers to any of the immunologic activities of 4B5, or to any other biological activity ascribed to 4B5 in this disclosure, including the role of 4B5 in the detection of antibodies specific for GD2, amelioration or palliation of cancer.

The “V region” of 4B5 refers to the V region of the 4B5 L chain or the V region of the 4B5 H chain, either alone or in combination. These V regions are depicted in SEQ ID NOS: 3 and 6; the DNA encoding these regions is depicted in SEQ ID NOS: 1 and 4, respectively.

GM-CSF, IL-2, and other biologically active molecules referred to herein are meant to include fragments and derivatives based on the respective parent molecule that have the same biologic or physiologic function.

A “polynucleotide” is a polymeric form of nucleotides of any length, which contain deoxyribonucleotides, ribonucleotides, and analogs in any combination analogs. Polynucleotides may have any three-dimensional structure, and may perform any function, known or unknown. The term “polynucleotide” includes double-, single-stranded, and triple-helical molecules. Unless otherwise specified or required, any embodiment of the invention described herein that is a polynucleotide encompasses both the double-stranded form and each of two complementary single-stranded forms known or predicted to make up the double stranded form of either the DNA, RNA or hybrid molecules.

The following are non-limiting examples of polynucleotides: a gene or gene fragment, exons, introns, MRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A polynucleotide can comprise modified nucleotides, such as methylated nucleotides and nucleotide analogs, uracyl, other sugars and linking groups such as fluororibose and thioate, and nucleotide branches. Analogs of purines and pyrimidines are known in the art, and include, but are not limited to, aziridinylcytosine, 4,acetylcytosine, 5-fluorouracil, 5-bromouracil, 5-carboxymethylaminomethyl-2-thiouracil, 5-carboxymethyl-aminomethyluracil, inosine, N6-isopentenyladenine, 1-methyladenine, 1-methylpseudouracil, 1-methylguanine, 1-methylinosine, 2,2dimethylguanine, 2-methyladenine, 2-methylguanine, 3-methylcytosine, 5-methylcytosine, pseudoruacil, 5-pentynyluracil and 2,6-diaminopurine. The use of uracil as a substitute for thymine in a deoxyribonucleic acid is also considered an analogous form of pyrimidine.

The sequence of nucleotides can be interrupted by non-nucleotide components. A polynucleotide can be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support.

If present, modification to the nucleotide structure can be imparted before or after assembly of the polymer. The sequence of nucleotides can be interrupted by non-nucleotide components. A polynucleotide can be further modified after polymerization, such as by conjugation with a labeling component. Other types of modifications included in this definition are, for example, “caps”, substitution of one or more of the naturally occurring nucleotides with an analog, intemucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s).

Further, any of the hydroxyl groups ordinarily present in the sugars may be replaced by phosphonate groups, phosphate groups, protected by standard protecting groups, or activated to prepare additional linkages to additional nucleotides, or may be conjugated to solid supports. The 5′ and 3′ terminal OH groups can be phosphorylated or substituted with amines or organic capping group moieties of from 1 to 20 carbon atoms. Other hydroxyls can also be derivatized to standard protecting groups.

Polynucleotides can also contain analogous forms of ribose or deoxyribose sugars that are generally known in the art, including, but not limited to, 2′-O-methyl-, 2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside.

As noted above, one or more phosphodiester linkages can be replaced by alternative linking groups. These alternative linking groups include, but are not limited to, embodiments wherein phosphate is replaced by P(O)S (“thioate”), P(S)S (“dithioate”), “(O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalky, cycloalkenyl or araldyl. Not all linkages in a polynucleotide need be identical.

Although conventional sugars and bases will be used in applying the method of the invention, substitution of analogous forms of sugars, purines and pyrimidines can be advantageous in designing a fmal product, as can alternative backbone structures like a polyamide backbone.

The term “recombinant” polynucleotide means a polynucleotide of genomic, cDNA, semisynthetic, or synthetic origin which either does not occur in nature or is linked to another polynucleotide in a nonnatural arrangement.

An “isolated” polynucleotide or polypeptide is one that is substantially free of the materials with which it is associated in its native environment. By substantially free is meant at least 50%, preferably at least 70%, more preferably at least 80%, and even more preferably at least 90% free of these materials.

A “stable duplex” of polynucleotides, or a “stable complex” formed between any two or more components in a biochernical reaction, refers to a duplex or complex that is sufficiently long-lasting to persist between the formation of the duplex or complex and subsequent detection, including any optional washing steps or other manipulation that may take place in the interim. -A “vector” refers to a recombinant DNA or RNA plasmid or virus that comprises a heterologous polynucleotide to be delivered into a target cell, either in vitro or in vivo. The heterologous polynucleotide may comprise a sequence of interest for purposes of therapy, and may optionally be in the form of an expression cassette. As used herein, a vector need not be capable of replication in the ultimate target cell or subject. The term includes cloning vectors for the replication of a polynucleotide, and expression vectors for translation of a polynucleotide encoding sequence. Also included are viral vectors, which comprise a polynucleotide encapsidated or enveloped in a viral particle.

The terms “polypeptide”, “peptide” and “protein” are used interchangeably herein to refer to polymers of amino acid residues of any length. The polymer may be linear or branched, it may comprise modified amino acids or amino acid analogs, and it may be interrupted by chemical moieties other than amino acids. The terms also encompass an amino acid polymer that has been modified naturally pr by intervention; for example, disulfide bond formation, glycosylation, lipidation, acetylation, phosphorylation, or any other manipulation or modification, such as conjugation with a labeling or bioactive component. Unless stated or implied otherwise, the term 4B5 includes any polypeptide monomer or polymer with 4B5 immunologic specificity, including the intact recombinant antibody, and smaller and larger functionally equivalent polypeptides.

A “fusion polypeptide” is a polypeptide comprising regions in a different position in the sequence than occurs in nature. The regions may normally exist in separate proteins and are brought together in the fusion polypeptide; they may normally exist in the same protein but are placed in a new arrangement in the fusion polypeptide; or they may be synthetically arranged. For instance, as described below, the invention encompasses recombinant proteins (and the polynucleotides encoding the proteins) that are comprised of a finctional portion of 4B5 and a toxin. Methods of making these fusion proteins are known in the art and are described for instance in WO93/07286.

A “functionally equivalent fragment” of a 4B5 polypeptide varies from the native sequence by any combination of additions, deletions, or substitutions while preserving at least one functional property of the fragment relevant to the context in which it is being used. A functionally equivalent fragment of a 4B5 polynucleotide either encodes a polypeptide that is fuctionally equivalent to 4B5 when produced by an expression system, or has similar hybridization specificity as a 4B5 polynucleotide when used in a hybridization assay. A finctionally equivalent fragment of a 4B5 polypeptide typically has one or more of the following properties: ability to bind antibodies specific for GD2; ability to generate antibodies specific at least one type of cancer cell in a specific manner; and an ability to elicit an immune response with a similar antigen specificity as that elicited by 4B5 or GD2.

A “cell line” or “cell culture” denotes bacterial, plant, insect or higher eukaryotic cells grown or maintained in vitro. The descendants of a cell may not be completely identical (either morphologically, genotypically, or phenotypically) to the parent cell. A Mab may be produced by a hybridoma or other cell. Methods of making hybridomas, both murine and human, are known in the art. Particular methods of producing human hybridomas are described and referenced throughout the specification.

A “host cell” denotes a prokaryotic or eukaryotic cell that has been genetically altered, or is capable of being genetically altered by administration of an exogenous polynucleotide, such as a recombinant plasmid or vector. When referring to genetically altered cells, the term refers both to the originally altered cell, and to the progeny thereof.

“Heterologous” means derived from a genotypically distinct entity from the rest of the entity to which it is being compared. For example, a polynucleotide may be placed by genetic engineering techniques into a plasmid or vector derived from a different source, and is a heterologous polynucleotide. A promoter removed from its native coding sequence and operatively linked to a coding sequence other than the native sequence is a heterologous promoter.

A “signal peptide” or “leader sequence” is a short amino acid sequence that directs a newly synthesized protein through a cellular membrane, usually the endoplasmic reticulum in eukaryotic cells, and either the inner membrane or both inner and outer membranes of bacteria. Signal peptides are typically at the N-terminal portion of a polypeptide and are typically removed enzymatically between biosynthesis and secretion of the polypeptide from the cell. The signal peptide is not present in the secreted protein, only during protein production.

A “biological sample” encompasses a variety of sample types, including blood and other liquid samples of biological origin, solid tissue samples such as a biopsy specimens or tissue cultures, or cells derived therefrom and the progeny thereof. The definition also includes samples that have been manipulated in any way after their procurement, such as by treatment with reagents, solubilization, or enrichment for certain components, such as proteins or polynucleotides. The term encompasses various kinds of clinical samples obtained from any species, and also includes cells in culture, cell supernatants, and cell lysates. Particularly, for the purposes described herein, biological samples comprise tumor tissue or tissue thought to be tumorous and are obtained for instance by surgical resection, biopsy, aspiration or any method known in the art.

An “immunogen” refers to composition for human or animal use, which is administered with the intention of conferring to the recipient a degree of specific immunologic reactivity against a particular antigen. The immunologic reactivity may be carried out by antibodies or cells (particularly B cells, plasma cells, T helper cells, and cytotoxic T lymphocytes, and their precursors) that are immunologically reactive against the target, or any combination thereof. For purposes of this invention, the target is primarily tumor-associated C antigen or a tumor-specific portion thereof. The immunologic reactivity may be desired for experimental purposes, for treatment of a particular condition, for the elimination of a particular substance, or for prophylaxis. An active immunogen is intended to elicit an immune response that persists in the absence of the vaccine components. “Adjuvant” as used herein has several meanings, all of which will be clear depending on the context in which the term is used. In the context of a pharmaceutical preparation, an adjuvant is a chemical or biological agent given in combination with or recombinantly fused to an antigen to enhance immunogenicity of the antigen. In the context of cancer diagnosis or management, adjuvant refers to a class of cancer patients with no clinically detectable tumor mass, but who are suspected of being at risk of recurrence.

When referring to a type of cancer that normally manifests as a solid tumor, a “clinically detectable” tumor is one that is detectable on the basis of tumor mass; i.e., by such procedures as CAT scan, X-Ray, or palpation. Biochemical, histological or immunologic findings alone may be insufficient to meet this definition.

As used herein, “treatment” refers to clinical intervention in an attempt to alter the natural course of the individual or cell being treated, and may be performed either for prophylaxis or during the course of clinical pathology. Desirable effects of the treatment include preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

The “pathology” associated with a disease condition is any condition that compromises the well-being, normal physiology, or quality of life of the affected individual. This may involve, but is not limited to, destructive invasion of affected tissues into previously unaffected areas, growth at the expense of normal tissue finction, irregular or suppressed biological activity, aggravation or suppression of an inflammatory or immunologic response, increased susceptibility to other pathogenic organisms or agents, and undesirable clinical symptoms such as pain, fever, nausea, fatigue, mood alterations, and such other features as may be determined by an attending physician.

An “effective amount” is an amount sufficient to effect a beneficial or desired clinical result. An effective amount can be administered in one or more doses. In terms of treatment, an effective amount is amount that is sufficient to palliate, ameliorate, stabilize, reverse or slow the progression of the disease, or otherwise reduce the pathological consequences of the disease. In terms of an adjuvant, an effective amount is one sufficient to enhance the immune response to the immunogen. The effective amount is generally determined by the physician on a case-by-case basis and is within the skill of one in the art. Several factors are typically taken into account when determining an appropriate dosage. These factors include age, sex and weight of the patient, the condition being treated, the severity of the condition and the form of the antibody being administered. For instance, the concentration of scFv need not be as high as that of native antibodies in order to be therapeutically effective.

An “individual”, “patient” or “subject” is a vertebrate, preferably a mammal, more preferably a human. Mammals include, but are not limited to, humans, farm animals, sport animals, and pets.

The practice of the present invention employs, unless otherwise indicated, conventional techniques of molecular biology (including recombinant techniques), microbiology, cell biology, biochemistry and immunology, which are within the skill of the art. Such techniques are explained fully in the literature, such as, “Molecular Cloning: A Laboratory Manual”, second edition (Sambrook et al., 1989); “Oligonucleotide Synthesis” (M.J. Gait, ed., 1984); “Animal Cell Culture” (R. I. Freshney, ed., 1987); “Methods in Enzymology” (Academic Press, Inc.); “Handbook of Experimental Immunology” (D. M. Wei & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction”, (Mullis et al., eds., 1994); “Current Protocols in Immunology” (J. E. Coligan et al., eds., 1991). These techniques are applicable to the production of the polynucleotides and polypeptides of the invention, and, as such, may be considered in making and practicing the invention. Particularly useful techniques for particular embodiments will be discussed in the sections that follow.

The invention also encompasses 4B5 conjugated to a chemically functional moiety. Typically, the moiety is a label capable of producing a detectable signal. These conjugated 4B5 are useful, for example, in detection systems such as quantitation of tumor burden, and imaging of metastatic foci and tumor imaging. Such labels are known in the art and include, but are not limited to, radioisotopes, enzymes, fluorescent compounds, chemiluminescent compounds, bioluminescent compounds substrate cofactors and inhibitors. See, for examples of patents teaching the use of such labels, U.S. Pat. Nos. 3,817,837; 3,850,752; 3,939,350; 3,996,345; 4,277,437; 4,275,149; and 4,366,241. The moieties can be covalently linked to 4B5, recombinantly linked, or conjugated to 4B5 through a secondary reagent, such as a second antibody, protein A, or a biotin-avidin complex.

Other functional moieties include signal peptides, agents that enhance immunologic reactivity, agents that facilitate coupling to a solid support, vaccine carriers, bioresponse modifiers, paramagnetic labels and drugs. Signal peptides are described above and include prokaryotic and eukaryotic forms. Agents that enhance immunologic reactivity include, but are not limited to, bacterial superantigens. Agents that facilitate coupling to a solid support include, but are not limited to, biotin or avidin. Immunogen carriers include, but are not limited to, any physiologically acceptable buffers. Bioresponse modifiers include cytokines, particularly tumor necrosis factor (TNF), interleukin-2, interleukin-4, granulocyte macrophage colony stimulating factor and γ interferons.

Suitable drug moieties include antineoplastic agents. These include, but are not limited to, tadioisotopes, vinca alkaloids such as the vinblastine, vincristine and vindesine sulfates, adriamycin, bleomycin sulfate, carboplatin, cispiatin, cyclophosphamide, cytarabine, dacarbazine, dactinomycin, duanorubicin hydrochloride, doxorubicin hydrochloride, etoposide, fluorouracil, lomustine, mechlororethamine hydrochloride, melphalan, mercaptopurine, methotrexate, mitomycin, mitotane, pentostatin, pipobroman, procarbaze hydrochloride, streptozotocin, taxol, thioguanine, and uracil mustard.

Immunotoxins, including single chain molecules, can be produced by recombinant means. Production of various immunotoxins is well-known in the art, and methods can be found, for example, in “Monoclonal Antibody-toxin Conjugates: Aiming the Magic Bullet,” Thorpe et al. (1982) Monoclonal Antibodies in Clinical Medicine, Academic Press, pp. 168-190; Vitatta (1987) Science 238:1098-1104; and Winter and Milstein (1991) Nature 349:293-299. Suitable toxins include, but are not limited to, ricin, radionuclides, pokeweed antiviral protein, Pseudomonas exotoxin A, diphtheria toxin, ricin A chain, fungal toxins such as restrictocin and phospholipase enzymes. See, generally, “Chimeric Toxins,” Olsnes and Pihl, Pharmac. Ther. 15:355-381 (1981); and “Monoclonal Antibodies for Cancer Detection and Therapy,” eds. Baldwin and Byers, pp. 159-179, 224-266, Academic Press (1985).

The chemically functional moieties can be made recombinantly for instance by creating a fusion gene encoding the antigen binding fragment and finctional regions from other genes (e.g. enzymes). In the case of gene fusions, the two components are present within the same polypeptide gene. Alternatively, the 4B5 antigen binding fragments can be chemically bonded to the moiety by any of a variety of well known chemical procedures. For example, when the moiety is a protein, the linkage may be by way of heterobifunctional cross linkers, e.g., SPDP, carbodiimide glutaraldehyde, or the like. The moieties may be covalently linked, or conjugated, through a secondary reagent, such as a second antibody, protein A, or a biotin-avidin complex. Paramagnetic moieties and the conjugation thereof to antibodies are well-known in the art. See, e.g., Miltenyi et al. (1990) Cytometry 11:231-238.

The 4B5 antibody of this invention can be prepared in several ways. It is most conveniently obtained from cells engineered to express the GD2 mimic fragment containing SEQ ID NOS: 1 and 4 or other polynucleotides encoding 4B5 binding fragments. For example, the cells can be cultured in a suitable medium, and spent medium can be used as an antibody source. Optionally, matrix-coated channels or beads and cell co-cultures may be included to enhance growth of antibody-producing cells. For the production of large amounts of antibody, it is generally more convenient to obtain an ascites fluid. The method of raising ascites generally comprises injecting hybridoma cells into an immunologically naive histocompatible or immunotolerant mammal, especially a mouse. The mammal may be primed for ascites production by prior administration of a suitable composition; e.g., Pristane.

Alternatively, 4B5 can be chemically synthesized using sequence data and other information provided in this disclosure, in conjunction with standard methods of protein synthesis. A suitable method is the solid-phase Merrifield technique. Automated peptide synthesizers are commercially available, such as those manufactured by Applied Biosystems, Inc. (Foster City, Calif.).

4B5 can also be obtained by employing routine recombinant methods such as described in Sambrook et al. (1989). For instance, using the amino acid and polynucleotide sequences provided herein and information provided herein, a polynucleotide encoding either the 4B5 H or L chain can be cloned into a suitable expression vector (which contains control sequences for transcription, such as a promoter). The expression vector is in turn introduced into a host cell. The host cell is grown under suitable conditions such that the polynucleotide is transcribed and translated into a protein. H and L chains of 4B5 can be produced separately, and then combined by disulfide bond rearrangement. Alternatively, vectors with separate polynucleotides encoding each chain of 4B5, or a vector with a single polynucleotide encoding both chains as separate transcripts, can be transfected into a single host cell which can then produce and assemble the entire molecule. Preferably, the host cell is derived from a higher eukaryote that can provide the normal carbohydrate complement of the molecule. The 4B5 thus produced can be purified using standard techniques in the art. Polynucleotides encoding 4BS for use in the production of 4B5 can be produced synthetically or recombinantly from the DNA sequences provided herein.

“Humanized” antibodies are antibodies in which at least part of the sequence has been altered from its initial form to render it more like human immunoglobulins. In one version, the H chain and L chain C regions are replaced with human sequence. This is a fusion polypeptide comprising a H11 V region and a heterologous immunoglobulin C region. In another version, the CDR regions comprise H11 amino acid sequences, while the V framework regions have also been converted human sequences. See, for example, EP 0329400. In a third version, V regions are humanized by designing consensus sequences of human and mouse V regions, and converting residues outside the CDRs that are different between the consensus sequences. The invention encompasses humanized Mabs.

In making humanized antibodies, the choice of framework residues can be critical in retaining high binding affinity. In principle, a framework sequence from any HuAb can serve as the template for CDR grafting; however, it has been demonstrated that straight CDR replacement into such a framework can lead to significant loss of binding affinity to the antigen. Glaser et al. (1992) J. Immunol. 149:2606; Tempest et al. (1992) Biotechnology 9:266; and Shalaby et al. (1992) J. Exp. Med. 17:217. The more homologous a HuAb is to the original muab, the less likely that the human framework will introduce distortions into the murine CDRs that could reduce affinity. Based on a sequence homology search against an antibody sequence database, the HuAb IC4 provides good framework homology to muM4TS.22, although other highly homologous HuAbs would be suitable as well, especially kappa L chains from human subgroup I or H chains from human subgroup III. Kabat et al. (1987). Various computer programs such as ENCAD (Levitt et al. (1983) J. Mol. Biol. 168:595) are available to predict the ideal sequence for the V region. The invention thus encompasses HuAbs with different V regions. It is within the skill of one in the art to determine suitable V region sequences and to optimize these sequences. Methods for obtaining antibodies wvith reduced immunogenicity are also described in U.S. Pat. No. 5,270,202 and EP 699,755.

Methods of antibody production and isolation are well known in the art. See, for example, Harlow and Lane (1988) Antibodies: A Laboratory Manual, Cold Spring Harbor Laboratory, New York. The 4B5 antibody is a human immunoglobulin of the IgG subclass, and may be isolated by any technique suitable for immunoglobulins of this isotype. Purification methods may include salt precipitation (for example, with ammonium sulfate), ion exchange chromatography (for example, on a cationic or anionic exchange column run at neutral pH and eluted with step gradients of increasing ionic strength), gel filtration chromatography (including gel filtration HPLC), and chromatography on affmity resins such as protein A, protein G, hydroxyapatite, and anti-immunoglobulin. 4B5 can also be purified on affinity columns comprising the anti-GD2 antibodies. Preferably, 4B5 is purified using Protein-A-CL-Sepharose™ 4B chromatography followed by chromatography on a DEAE-Sepharose™ 4B ion exchange column.

The invention also encompasses hybrid antibodies, in which one pair of H and L chains is obtained from a first antibody, while the other pair of H and L chains is obtained from a different second antibody. For purposes of this invention, one pair of L and H chains is from 4B5. In one example, each L-H chain pair binds different epitopes of the 4B5 antigen. Such hybrids can also be formed using humanized H or L chains.

Another 4B5 contemplated by this invention is an antibody in which the H or L chain has been modified to provide additional properties. For instance, a change in amino acid sequence can result in reduced immunogenicity of the resultant polypeptide. The changes range from changing of one or more amino acids t the complete redesign of a region such as a C region domain Typical changes include, but are not limited to, those related to complement fixation, interaction with membrane receptors, and other effector functions. Also encompassed by the invention are peptides in which various immunoglobulin domains have been placed in an order other than that which occurs in nature.

If 4B5 is to be administered to an individual, it is preferably at least 80% pure, more preferably it is at least 90% pure, even more preferably it is at least 95% pure and free of pyrogens and other contaminants. In this context, the percent purity is calculated as a weight percent of the total protein content of the preparation, and does not include constituents which are deliberately added to the composition after the 4B5 is purified.

Polynucleotides of the Invention

The invention provides various polynucleotides encoding the antibody 4B5 or fragments of 4B5, based on the polynucleotide sequences provided herein (SEQ ID NOS: 1 and 4) and the complimentary polynucleotides (SEQ ID NOS: 2 and 6). Various embodiments are described in this section, comprising a number of different combinations of the 4B5 H or L chain V region sequences. In general, a 4B5 polynucleotide of this invention encodes at least one feature that is unique to the 4B5 molecule (in comparison with other immunoglobulins). Preferably, this feature is related in some way to an immunologic reactivity of 4B5.

The invention encompasses polynucleotides encoding a portion of the 4B5 L chain V region, comprising at least about 70 consecutive nucleotides, preferably at least about 80 consecutive nucleotides, more preferably at least about 100 consecutive nucleotides, even more preferably at least about 150 nucleotides of SEQ ID NO: 4. The invention also encompasses a polynucleotide encoding a portion of the 4B5 L chain V region, comprising at least about 25 consecutive nucleotides, preferably at least about 30 consecutive nucleotides, and even more preferably at least abut 35 consecutive nucleotides of the CDRI encoding sequence thereof. The invention also encompasses a polynucleotide encoding a portion of the 4B5 L chain V region, comprising at least about 20 consecutive nucleotides, preferably at least about 25 consecutive nucleotides, and even more preferably at least about 35 consecutive nucleotides of the CDR2 or CDR3 encoding sequence thereof.

The invention also encompasses polynucleotides encoding a portion of the 4B5 H chain V region, comprising at least about 70 consecutive nucleotides, preferably at least about 80 consecutive nucleotides, more preferably at least about 100 consecutive nucleotides, even more preferably at least about 150 nucleotides of SEQ ID NO: 1. The invention also encompasses a polynucleotide encoding a portion of the 4B5 L chain V region, comprising 15 consecutive nucleotides of the CDRI encoding sequence thereof. The invention also encompasses a polynucleotide encoding a portion of the 4B5 L chain V region, comprising at least about 20 consecutive nucleotides, preferably at least about 25 consecutive nucleotides, and even more preferably at least about 35 consecutive nucleotides of the CDR2 or CDR3 coding sequence thereof.

The invention includes isolated 4B5 polynucleotides encoding a polypeptide having immunologic activity of 4B5, wherein the polypeptide encodes at least 5 amino acids of a V L chain of 4B5 as depicted in SEQ ID NO: 5. The invention also includes isolated 4B5 polynucleotides encoding a polypeptide having immunologic activity of 4B5, wherein the polynucleotide encodes at least 5 amino acids of a V H chain of 4B5 as depicted in SEQ ID NO: 3. The polynucleotide sequence can be similar to those depicted in SEQ ID NO: 1 or SEQ ID NO: 4 with changes designed to optimize codon usage, stability, facilitate cloning, or any other purpose. It is within the skill of one in the art, given the amino acid sequence in SEQ ID NO: 3 or SEQ ID NO: 6, to design such polynucleotides. Preferred polynucleotides encode at least five amino acids of a 4B5 CDR.

The invention also encompasses polynucleotides encoding for flnctionally equivalent variants and derivatives of 4B5 and functionally equivalent fragments thereof which may enhance, decrease or not significantly affect properties of the polypeptides encoded thereby. These fumctionally equivalent variants, derivatives, and fragments display the ability to specifically recognize C antigen. For instance, changes in a DNA sequence that do not change the encoded amino acid sequence, as well as those that result in conservative substitutions of amino acid residues, one or a few amino acid deletions or additions, and substitution of amino acid residues by amino acid analogs are those which will not significantly affect properties of the encoded polypeptide. Conservative amino acid substitutions are glycine/alanine; valine/isoleucine/leucine; asparagine/glutamine; aspartic acid/glutamic acid; serine/threonine/methionine; lysine/arginine; and phenylalanine/tyrosine/tryptophan.

The polynucleotides of the invention can comprise additional sequences, such as additional encoding sequences within the same transcription unit, controlling elements such as promoters, ribosome binding sites, and polyadenylation sites, additional transcription units under control of the same or a different promoter, sequences that permit cloning, expression, and transformation of a host cell, and any such construct as may be desirable to provide embodiments of this invention.

The invention encompasses a polynucleotide of at least about 15 consecutive nucleotides, preferably at least about 20 nucleotides, more preferably at least about 25 consecutive nucleotides, more preferably at least about 35 consecutive nucleotides, more preferably at least about 50 consecutive nucleotides, even more preferably at least about 75 nucleotides, still more preferably at least about 100 nucleotides, still more preferably at least about 200 nucleotides, and even more preferably at least about 300 nucleotides that forms a stable hybrid with a polynucleotide encoding the L chain or H chain V region of 4B5, but not with other immunoglobulin encoding regions known at the time of filing of this application. Any set of conditions may be used for this test, as long as at least one set of conditions exist wherein the test polynucleotide demonstrates the required specificity. Preferably, the 4B5 encoding sequences to which the test polynucleotide binds are those shown in SEQ ID NOS: 2 and 5. Since the known immunoglobulin sequences fall into a hierarchy of similarity with that of 4B5, the test may be performed by comparing the hybridization of the test polynucleotide with the 4B5 sequence with the hybridization with the most closely related sequences. Preferred is a panel of about 10 of the sequences most closely related to SEQ ID NO: 1 or 4.

Hybridization reactions can be performed under conditions of different “stringency”. Conditions that increase stringency of a hybridization reaction are well known. See, for example, Sambrook and Maniatis. Examples of relevant conditions include (in order of increasing stringency): incubation temperatures of 25° C., 37° C., 50° C. and 68° C.; buffer concentrations of 10×SSC, 6×SSC, 1×SSC, 0.1×SSC (where SSC is 0.15M NaCl and 15 mM citrate buffer) and their equivalent using other buffer systems; formamide concentrations of 0%, 25%, 50%, and 75%; incubation times from 5 minutes to 24 hours; 1, 2, or more washing steps; wash incubation times of 1, 2, or 15 minutes; and wash solutions of 6×SSC, 1×SSC, 0.1×SSC, or deionized water.

Useful 4B5 polynucleotides encoding fragments of 4B5 can be identified by generating polynucleotide fragments (based on SEQ ID NO: 1 or SEQ ID NO: 4, for example) and testing the polypeptides encoded thereby for a function of interest. Alternatively, the polypeptide fragment encoded by a particular polynucleotide can be prepared and tested for a function of interest. Alternatively, given a 4B5 polypeptide with desirable properties, polynucleotides can be designed that encode the polypeptide.

Included in all these embodiments are polynucleotides with encoding regions for 4B5 polymers, fusion proteins, humanized immunoglobulins, single-chain V regions, and other particular polypeptides of interest. These polypeptides are described above.

The invention also provides polynucleotides covalently linked with a detectable label. Such polynucleotides are useful, for example, as probes for detection of related nucleotide sequences.

The polynucleotides of this invention can be obtained using chemical synthesis, recombinant cloning methods, PCR, or any combination thereof. Methods of chemical polynucleotide synthesis are well known in the art and need not be described in detail herein. One of skill in the art can use the sequence data provided herein to obtain a desired polynucleotide by employing a DNA synthesizer or ordering from a commercial service.

Polynucleotides comprising a desired sequence can be inserted into a suitable vector, and the vector in turn can be introduced into a suitable host cell for replication and amplification. Polynucleotides can be introduced into host cells by any means known in the art. Cells are transformed by introducing an exogenous polynucleotide by direct uptake, endocytosis, transfection, f-mating or electroporation. Once introduced, the exogenous polynucleotide can be maintained within the cell as a non-integrated vector (such as a plasmid) or integrated into the host cell genome. Amplified DNA can be isolated from the host cell by standard methods. See, e.g., Sambrook et al. (1989). RNA can also be obtained from transformed host cell, or it can be obtained directly from the DNA by using a DNA-dependent RNA polymerase.

The present invention further encompasses a variety of vectors comprising a 4B5 polynucleotide. These vectors can be used for expression of recombinant polypeptides are also a source of 4B5 polynucleotides. Cloning vectors can be used to obtain replicate copies of the 4B5 polynucleotides they contain, or as a means of storing the polynucleotides in a depository for future recovery. Expression vectors (and host cells containing these expression vectors) can be used to obtain polypeptides produced from the polynucleotides they contain. They can also be used where it is desirable to express 4B5 in an individual and thus have intact cells capable of synthesizing the polypeptide, such as in gene therapy. Suitable cloning and expression vectors include any known in the art, e.g., those for use in bacterial, mammalian, yeast and insect expression systems. Specific vectors and suitable host cells are known in the art and are not described in detail herein. See e.g. Gacesa and Ramji Vectors, John Wiley & Sons (1994).

Cloning and expression vectors typically contain a selectable marker (for example, a gene encoding a protein necessary for the survival or growth of a host cell transformed with the vector), although such a marker gene can be carried on another polynucleotide sequence co-introduced into the host cell. Only those host cells into which a selectable gene has been introduced will grow under selective conditions. Typical selection genes either: (a) confer resistance to antibiotics or other toxins, e.g., ampicillin, neomycin, methotrexate; (b) complement auxotrophic deficiencies; or (c) supply critical nutrients not available from complex media. The choice of the proper marker gene will depend on the host cell, and appropriate genes for different hosts are known in the art. Vectors also typically contain a replication system recognized by the host.

Suitable cloning vectors can be constructed according to standard techniques, or selected from a large number of cloning vectors available in the art. While the cloning vector selected may vary according to the host cell intended to be used, useful cloning vectors will generally have the ability to self-replicate, may possess a single target for a particular restriction endonuclease, or may carry marker genes. Suitable examples include plasmids and bacterial viruses, e.g., pUC18, mp18, mp19, pBR322, pMB9, ColE1, pCR1, RP4, phage DNAs, and shuttle vectors such as pSA3 and pAT28. These and other cloning vectors are available from commercial vendors such as BioRad, Stratagene, and Invitrogen.

Expression vectors generally are replicable polynucleotide constructs that contain a polynucleotide encoding a 4B5 polypeptide of interest. The polynucleotide encoding 4B5 polypeptide is operatively linked to suitable transcriptional controlling elements, such as promoters, enhancers and terminators. For expression (i.e., translation), one or more translational controlling elements are also usually required, such as ribosome binding sites, translation initiation sites, and stop codons. These controlling elements (transcriptional and translational) can be derived from the 4B5 gene, or heterologous (i.e., derived from other genes or other organisms). A polynucleotide sequence encoding a signal peptide can also be included to allow a 4B5 polypeptide to cross or lodge in cell membranes or be secreted from the cell. A number of expression vectors suitable for expression in eukaryotic cells including yeast, avian, and mammalian cells are known in the art. One example of an expression vector is pcDNA3 (Invitrogen, San Diego, Calif.), in which transcription is driven by the cytomegalovirus (CMV) early promoter/enhancer. This vector also contains recognition sites for multiple restriction enzymes for insertion of an 4B5 polynucleotide of interest. Another example of an expression vector (system) is the baculovirus/insect system.

Also encompassed by the invention are expression systems suitable for use in antibody-targeted gene therapy comprising a 4B5 polynucleotide. Suitable systems are described for instance by Brown et al. (1994) Virol. 198:477-488; and Miyamura et al. (1994) Proc. Natl. Acad. Sci USA 91:8507-8511.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (where the vector is an infectious agent, such as vaccinia virus, which is discussed below). The choice of introducing vectors or 4B5 polynucleotides will often depend on features of the host cell.

Once introduced into a suitable host cell, expression of a 4B5 polypeptide can be determined using any assay known in the art. For example, presence of 4B5 polypeptide can be detected by RIA or ELISA of the culture supernatant (if the 4B5 polypeptide is secreted) or cell lysates.

A particularly useful expression vector for 4B5 polynucleotides is a vaccinia virus comprised of a 4B5 polynucleotide sequence, which can also be used in vaccine preparations. Moss (1991) Science 252:1662-1667. To introduce polynucleotide sequences encoding 4B5 polypeptide, including 4B5 polypeptide fragments, into vaccinia, the polynucleotide sequence of interest is first inserted into a plasmid containing a vaccinia virus promoter with flanking sequences homologous to vaccinia DNA not required for replication. Plasmid-containing cells are then infected with vaccinia, which leads to a low level of homologous recombination between plasmid and virus, with resultant transfer of the vaccinia promoter and 4B5 polypeptide-encoding polynucleotide sequence into the vaccinia virus genome. Typically, the 4B5 polynucleotide is inserted into the viral TK (thymidine kinase) gene. Insertion into the TK site attenuates the virus more than 10,000 fold compared to wild type. Flexner et al. (1980) Vaccine 88 (Cold Spring Harbor Laboratory), pp. 179-184. Recombinant virus is identified by the TK⁻ phenotype. Preferably, expression of the 4B5 polynucleotide is under the control of the vaccinia early/late promoter (7.5 K), whereby the resultant 4B5 polypeptides can be expressed in infected cells throughout the life cycle of the virus. However, other promoters known in the art can be used, such as pH6, or synthetic promoters. Expression of the 4B5 polypeptide occurs in cells infected with the recombinant vaccinia or individuals immunized with the live recombinant vaccinia virus. Any one of several strains of vaccinia can be used, including, but not limited to, WR, ALVAC, and NYVAC.

A vector of this invention can contain one or more polynucleotides encoding a 4B5 polypeptide. It can also contain polynucleotide sequences encoding other polypeptides that enhance, facilitate, or modulate the desired result, such as lymphokines, including, but not limited to, IL-2, IL-4, GM-CSF, TNF-α, and IFN-γ. A preferred lymphokine is GM-CSF. Preferred GM-CSF constructs are those which have been deleted for the AU-rich elements from the 3′ untranslated regions and sequences in the 5′ untranslated region that are capable of forming a hairpin loop. Also embodied in this invention are vaccinia vectors encoding for recombinant 4B5 variants, such as scFvs, chimeras, and polymers.

Other embodiments of this invention are host cells transformed with 4B5 polynucleotides and vectors comprising 4B5 polynucleotide sequences, as described above. Both prokaryotic and eukaryotic host cells may be used. Prokaryotic hosts include bacterial cells, for example E. coli and Mycobacteria. Among eukaryotic hosts are yeast, insect, avian, plant and mammalian cells. Host systems are known in the art and need not be described in detail herein. Examples of mammalian host cells include CHO and NSO, obtainable from the European Collection of Cell Cultures (England). Transfection of NSO cells with a plasmid, for example, which is driven by a CMV promoter, followed by amplification of this plasmid in using glutamine synthetase provides a useful system for protein production. Cockett et al. (1990) Bio. Technology 8:662-667.

The host cells of this invention can be used, inter alia, as repositories of 4B5 polynucleotides, or as vehicles for production of 4B5 polynucleotides and polypeptides. They may also be used as vehicles for in vivo expression of 4B5 polypeptides. The 4B5 polynucleotides of this invention can be used in expression systems to produce 4B5 polypeptides, intact 4B5, or recombinant forms of 4B5, such as are described below.

The polynucleotides of this invention have several uses. They are useful, for example, in expression systems for the production of 4B5. They are also useful as hybridization probes to assay for the presence of 4B5 polynucleotide or related sequences in a sample using methods well known to those in the art. Further, the polynucleotides are also useful as primers to effect amplification of desired polynucleotides. The polynucleotides of this invention are also useful in pharmaceutical compositions including vaccines and for gene therapy.

The polynucleotides can also be used as hybridization probes for detection of 4B5 encoding sequences. Suitable samples include cells transformed ex vivo for use in gene therapy. In one illustration, DNA or RNA is extracted from a sample, and optionally run on a gel and/or digested with restriction endonucleases. The processed sample polynucleotide is typically transferred to a medium suitable for washing. The sample polynucleotide is then contacted with the 4B5 polynucleotide probe under conditions that permit a stable duplex to form if the sample contains a matching 4B5 sequence. Any stable duplexes formed are detected by any suitable means. For example, the 4B5 polynucleotide probe can be supplied in labeled form, and label remaining with the'sample after washing will directly reflect the amount of stable duplex formed. In a second illustration, hybridization is performed in situ. A suitably prepared tissue sample is overlaid with a labeled probe to indicate the location 4B5 encoding sequences.

A short 4B5 polynucleotide can also be used as a prnmer for a PCR reaction, particularly to amplify a longer sequence comprising a region hybridizing with the primer. This can be conducted preparatively, in order to produce polynucleotide for further genetic manipulation. It can also be conducted analythcally, to determine whether a 4B5 encoding polynucleotide is present, for example, in a sample of diagnostic interest.

Another use of the polynucleotides is in vaccines and gene therapy. The general principle is to administer the polynucleotide so that it either promotes or attenuates the expression of the polypeptide encoded therein. Thus, the present invention includes methods of inducing an immune response and methods of treatment comprising administration of an effective amount 4B5 polynucleotides to an individual. In these methods, a 4B5 polynucleotide encoding a 4B5 polypeptide is administered to an individual, either directly or via cells transfected with the 4B5 polynucleotide. Preferably, the 4B5 polynucleotide is in the form of a circular plasmid, preferably in a supercoiled configuration. Preferably, the 4B5 polynucleotide is replicated inside a cell. Thus, the 4B5 polynucleotide is operatively linked to a suitable promoter, such as a heterologous promoter that is intrinsically active in cells of the target tissue type. Preferably, once in cell nuclei, plasmids persist as circular non-replicating episomal molecules. In vitro mutation can be carried out with plasmid constructs to encode, for example, molecules with greater immunogenicity.

To determine whether plasmids containing 4B5 polynucleotides are capable of 4B5 expression in eukaryotic cells, cells such as COS-7, CHO, or HeLa can be transfected with the plasmids. Expression of 4B5 is then determined by immunoassay; for example, by Western blot. Smaller 4B5 polypeptides can be detected, for example, by constructing the plasmid so that the resultant 4B5 polypeptide is fused with a tag, such as a target epitope or enzyme label. Further characterization of the expressed 4B5 polypeptide can be achieved by purifying the peptide and then conducting one of the functional assays described herein.

In one mode of gene therapy, the polynucleotides of this invention are used for genetically altering cells ex vivo. In this strategy, cells removed from a donor or obtained from a cell line are transfected or transduced with vectors encoding a 4B5 polypeptide, and then administered to a recipient. Suitable cells for transfection include peripheral blood mononuclear cells.

In another mode of gene therapy, the polynucleotides of this invention are used for genetically altering cells in vivo. The purpose includes, but is not limited to, treating various types of cancer.

Polypeptides of the Invention

The invention also encompasses polypeptide fragments of 4B5 containing at least a portion of a V region of 4B5. Preferred fragments are those with the immunogenic activity of 4B5. Also preferred are fragments which comprise amino acid sequences substantially different from other immunoglobulins, and fragments comprising a CDR. In one embodiment, the invention includes a polypeptide fragment of the 4B5 H chain V region, comprising at least 25 consecutive amino acids, more preferably 30 consecutive amino acids of SEQ ID NO: 3, or 5 consecutive amino acids of the CDR1 thereof, or at least 7 consecutive amino acids, preferably at least 9 consecutive amino acids of the CDR2 or CDR3 thereof. The invention also includes a polypeptide fragment of the 4B5 L chain V region, comprising at least 25 consecutive amino acids, more preferably 30 consecutive amino acids of SEQ ID NO: 6, or 7 consecutive amino acids of the CDR2 thereof, or at least 8 consecutive amino acids, preferably 10 consecutive amino acids of the CDR1 or CDR3 thereof.

The size of the 4B5 polypeptides can be only the minimum size required to provide a desired finction. The polypeptides can optionally comprise additional sequence, either native to 4B5, or from a heterologous source, as desired. 4B5 peptides can contain only 5 consecutive amino acids from a 4B5 V region. Polypeptides comprising 7 amino acids, more preferably about 10 amino acids, more preferably about 15 amino acids, more preferably about 25 amino acids, more preferably about 50 amino acids, more preferably about 75 amino acids from the 4B5 L or H chain V region are also included. Even more preferred are polypeptides comprising the entire 4B5 L or H chain V region. Preferably the polypeptides are derived from 4B5.

The invention includes modified 4B5 polypeptides which are functionally equivalent to 4B5, or have altered but measurable 4B5 immunogenic activity. Modified polypeptides with improved 4B5 immunogenic activity are preferred. Examples of modified polypeptides include those with conservative substitutions of amino acid residues, and one or more deletions or additions of amino acids which do not significantly deleteriously alter the immunologic activity.

One example of this is 4B5 polypeptides comprising one or more amino acid substitution in comparison with the prototype 4B5 sequence. Substitutions can range from changing or modifying one or more amino acid residues to complete redesign of a region, such as the V region. Amino acid substitutions, if present, are preferably conservative substitutions that do not deleteriously affect folding or finctional properties of the peptide. Groups of finctionally related amino acids within which conservative substitutions can be made are glycine/alanine; valine/isoleucine/leucine; asparagine/glutamine; aspartic acid/glutamic acid; serine/threonine/methionine; lysine/arginine; and phenylalanine/tryosine/tryptophan. Polypeptides of this invention can be in glycosylated or unglycosylated form, can be modified post-translationally (e.g., acetylation, and phosphorylation) or can be modified synthetically (e.g., the attachment of a labeling group).

4B5 polypeptide derivatives comprising both a 4B5 L chain and a 4B5 H chain can be formed as separate L and H chains and then assembled, or assembled in situ by an expression system for both chains. Such expression systems can be created by transfecting a suitable cell with a plasmid comprising separate transcribable regions for the L and H chain, or by co-transfecting the same cell with plasiids for each chain. In a third method, a suitable plasmid with a H chain encoding region is transfected into a H chain loss mutant.

H chain loss mutants can be obtained by treating approximately 2×10⁷ 4B5 producing cells with fluorescein-labeled rabbit anti-mouse IgG (H chain specific, DAKO Corporation, Carpinteria, Calif.) according to the supplier's instructions. The stained and unstained cell populations are analyzed in a fluorescence-activated cell sorter. The unstained cells are collected in a sterilized tube and placed in 96-well plates with 1 cell/well by limiting dilution. The culture supernatants are then assayed by ELISA using goat anti-mouse IgG (H chain specific) and goat anti-mouse kappa. The clones with kappa-positive and IgG-negative phenotype are subcloned at least 3 times to obtain stable 4B5^{(-H)} mutants. MRNA from putative H chain loss mutant 4B5^{(-H)} clones can be isolated and the sequence of the L chain V region cDNA determined. Reverse PCR of the MRNA for the 4B5 VH is performed with 2 sets of 5′- and 3′- primers, used for cloning of 4B5^{(-H)} cDNA (Example 7). A H chain loss mutant yields no detectable DNA band. Transfection of the cells proceeds with a suitable H chain plasmid.

Another 4B5 derivative encompassed by this invention is an antibody in which the 4B5 H or L chain has been modified to provide additional properties. For instance, a change in amino acid sequence can result in greater immunogenicity of the resultant polypeptide. The changes range from changing of one or more amino acids to the complete redesign of a region such as a 4B5 region domain. Changes contemplated affect complement fixation, interaction with membrane receptors, and other effector functions. Also encompassed by the invention are proteins in which various immunoglobulin domains have been placed in an order other than that which occurs in nature.

The invention also encompasses single chain V region fragments (“scFv”) of 4B5. Single chain V region fragments are made by linking L and/or H chain V regions by using a short linking peptide. Bird et al. (1988) Science 242:423-426. Any peptide having sufficient flexibility and length can be used as a linker in a scFv. Usually the linker is selected to have little to no immunogenicity. An example of a linking peptide is (GGGGS)₃, which bridges approximately 3.5 nm between the carboxy terminus of one V region and the amino terminus of another V region. Other linker sequences can also be used, and can provide additional functions, such as a means for attaching a drug or a solid support.

All or any portion of the H or L chain can be used in any combination. Typically, the entire V regions are included in the scFv. For instance, the L chain V region can be linked to the H chain V region. Alternatively, a portion of the L chain V region can be linked to the H chain V region, or a portion thereof. Also contemplated are scFvs in which the H chain V region is from 4B5, and the L chain V region is from another immunoglobulin. It is also possible to construct a biphasic, scFv in which one component is a 4B5 polypeptide and another component is a different polypeptide, such as a T cell epitope.

The scFvs can be assembled in any order, for example, V_{H}—(linker)—V_{L} or V_{L}—(linker)—V_{H}. Tandem scFvs can also be made, such as (X)—(linker)—(X)—(linker)—(X), in which X are 4B5 polypeptides, or combinations of 4B5 polypeptides with other polypeptides. In another embodiment, single chain antibody polypeptides have no linker polypeptide, or just a short, inflexible linker. Exemplary configurations include V_{L}—V_{H} and V_{H}—V_{L}. The linkage is too short to permit interaction between V_{L} and V_{H} within the chain, and the chains form homodimers with a V_{L}/V_{H} antigen binding site at each end. Such molecules are referred to in the art as “diabodies”.

ScFvs can be produced either recombinantly or synthetically. For synthetic production of scFv, an automated synthesizer can be used. For recombinant production of scFv, a suitable plasmid containing a polynucleotide that encodes the scFv can be introduced into a suitable host cell, either eukaryotic, such as yeast, plant, insect or mammalian cells, or prokaryotic, such as E. coli, and the protein expressed by the polynucleotide can be isolated using standard protein purification techniques.

A particularly useful system for the production of scFvs is plasmid pET-22b(+) (Novagen, Madison, Wis.) in E. coli. pET-22b(+) contains a nickel ion binding domain consisting of 6 sequential histidine residues, which allows the expressed protein to be purified on a suitable affinity resin. Another example of a suitable vector is pcDNA3 (Invitrogen, San Diego, Calif.), described above.

Repression conditions should ensure that the scFv assumes functional and, preferably, optimal tertiary structure. Depending on the plasmid used (especially the activity of the promoter) and the host cell, it may be necessary to modulate the rate of production. For instance, use of a weaker promoter, or expression at lower temperatures, may be necessary to optimize production of properly folded scFv in prokaryotic systems; or, it may be preferably to express scFv in eukaryotic cells.

Preferred scFv comprise at least 10 consecutive amino acids of SEQ ID NO: 3 and at least 10 consecutive amino acids of SEQ ID NO: 6, especially wherein the amino acids of SEQ ID NO: 3 and the amino acids of SEQ ID NO: 6 are joined by a linker polypeptide of 5 to 20 amino acids, or comprising the L chain V region and the H chain V region of 4B5.

The invention also encompasses polymeric forms of 4B5 polypeptides, containing a plurality of 4B5 polypeptides. One embodiment is a linear polymer of 4B5 polypeptides, optionally conjugated to carrier. These linear polymers can comprise multiple copies of a single 4B5 polypeptide, or combinations of different 4B5 polypeptides, and can have tandem 4B5 polypeptides, or 4B5 polypeptides separated by other amino acid sequences. Another embodiment is 4B5 multiple antigen peptides (MAPs). MAPs have a small immunologically inert core having radially branching lysine dendrites, onto which a number of 4B5 polypeptides are covalently attached. See for instance, Posnett et al. (1988) J. Biol. Chem. 263:1719-1725; and Tam (1989) Meth. Enz. 168:7-15. The result is a large macromolecule having a high molar ratio of 4B5 polypeptides to core. MAPs are efficient immunogens and useful antigens for immunoassays. The core for creating an 4B5 MAP can be made by standard peptide synthesis techniques, or obtained commercially, e.g., from Quality Controlled Biochemicals, Inc., Hopkinton, Mass. A typical core matrix is made up of three levels of lysine and eight amino acids.

When using 4B5 polypeptides as immunogens, preferably the polypeptides are delivered in conjunction with a carrier. Any carrier can be used which is not harmfu; to the host. Suitable carriers are typically large, slowly metabolized macromolecules such as proteins; polysaccharides (such as latex functionalized Sepharose, agarose, cellulose, cellulose beads and the like); polymeric amino acids (such as polyglutamic acid, polylysine, and the like); amino acid copolymers; and inactive virus particles or attenuated bacteria, such as Salmonella. Especially useful carrier proteins are serum albumins, keyhole limpet hemacyanin (KLH), certain Ig molecules, thyroglobulin, ovalbumin, and tetanus toxoid. KLH is especially preferred.

4B5 polypeptides of the invention can be identified in a number of ways. For example, the V regions of the L and H chains can be screened by preparing a series of short polypeptides that together span the entire V region amino acid sequence and determining whether they are recognized specifically by antibodies specific for GD2. Using a series of polypeptides of 20 or 50 amino acids in length, each 4B5 V region can be surveyed for useful functional properties. It is also possible to carry out a computer analysis of a protein sequence to identify potentially interesting polypeptides, such as those that bear the shape of GD2, or those involved in idiotype-anti-idiotype contact.

The invention further encompasses various adaptations of 4B5 described in this section combined in various fashions to yield other 4B5 polypeptides with desirable properties. For instance, 4B5 polypeptides with modified amino acid residues can be comprised in a MAP. In another example, a 4B5 scFv is fused to a cytokine, such as IL-2. All such combinations are contemplated in this invention.

The polypeptides of this invention can be made by any suitable procedure, including proteolysis of the 4B5 antibody, by recombinant methods or by chemical synthesis. These methods are known in the art and need not be described in detail herein. Examples of proteolytic enzymes include, but are not limited to, trypsin, chymotrypsin, pepsin, papain, V8 protease, subtilisin, plasmin, and thrombin. Intact 4B5 can be incubated with one or more proteinases simultaneously or sequentially. Alternatively, or in addition, intact antibodyican be treated with disulfide reducing agents. Peptides can then be separated from each other by techniques known in the art including, but not limited to, gel filtration chromatography, gel electrophoresis, and reverse-phase HPLC.

4B5 polypeptides can also be made by expression from a polynucleotide encoding the peptide according to the information provided elsewhere in this application, in a suitable expression system. Typically, polynucleotides encoding a 4B5 polypeptide are ligated into an expression vector under control of a suitable promoter and used to genetically alter the intended host cell. Both eukaryotic and prokaryotic host systems can be used. The polypeptide is then isolated from lysed cells or from the culture medium and purified to the extent needed for its intended use. Examples of prokaryotic host cells appropriate for use with this invention include E. coli. Examples of eukaryotic host cells include avian, insect, plant, and animal cells including, but not limited to, COS7, HeLa, and CHO cells.

In certain applications. such as when a 4B5 polypeptide is expressed in a suitable storage medium such as a plant seed, the 4B5 polypeptide can be used without purification. Fiedler et al. (1995) Biotechnology 13:1090-1093. For most applications, it is generally preferable that the polypeptide is at least partially purified from other cellular constituents. Preferably, the polypeptide is at least about 50% pure as a weight percent of total protein. More preferably, the protein is at least about 50-75% pure. For clinical use, the polypeptide is preferably at least about 80% pure.

4B5 polypeptides can be characterized in several ways. For instance, a 4B5 polypeptide may be tested for its ability to bind specifically to antibodies specific for GD2, or for its ability to specifically inhibit the binding between cancer cells and antibodies specific for GD2. A 4B5 polypeptide can also react with anti-CDR3 polypeptides. 4B5 polypeptides can also be tested for their ability to palliate or ameliorate neoplastic disease, such as carcinomas. It is understood that only one of these properties need be present in order for a polypeptide to come within the scope of this invention, although preferably more than one of these properties is present.

The ability of a 4B5 polypeptide to bind specific antibodies can be tested by immunoassay. Any form of direct binding assay is suitable. In one such assay, the antibody is labeled. Suitable labels include radioisotopes such as ¹²⁵I, enzymes such as peroxidase, fluorescent labels such as fluorescein, and chemiluminescent labels. Typically, the other binding partner is insolubilized (for example, by coating onto a microtiter plate) to facilitate washing. After combining the labeled component with the insolubilized component, the solid phase is washed and the amount of bound label is determined. Another such assay is a sandwich assay, in which the putative 4B5 polypeptide is captured by a first anti-immunoglobulin on a solid phase and developed with 4B5 antibody. In either of these examples, the extent of binding of 4B5 is directly related to the amount of label bound to the solid phase.

To conduct the inhibition assays, the putative 4B5 polypeptide is titered for its ability to decrease the binding of antibodies specific for GD2 to GD2. Typically, the anti-GD2 antibodies are labeled, while the GD2 is typically insolubilized in order to facilitate washing. The putative 4B5 polypeptide is typically mixed with the anti-GD2 antibodies and the mixture is combined with the solid phase. Polypeptides with the characteristics of 4B5 will proportionately decrease the amount of label attached to the solid phase, compared with control polypeptides. This test may be more sensitive than measuring direct binding, because lower affinity interaction between 4B5 and anti-GD2 antibodies may be too weak to form a stable bond, but adequate to interfere with the binding of another ligand-receptor pair when present at sufficient concentration.

The invention also encompasses methods of detecting antibodies specific for GD2 in a biological sample. The methods include obtaining a biological sample, contacting the sample with 4B5 under conditions that allow antibody antigen binding and detecting binding, if any, of 4B5 with the anti-GD2 antibodies.

When using intact murine 4B5, it is generally preferable to deplete the sample of any anti-mouse immunoglobulin activity that may be present. Anti-mouse immunoglobulin antibody can be removed from a sample, for example, by precipitation with normal mouse IgG or adsorption with a mouse Ig adsorbent. Binding of anti-mouse immunoglobulin antibody, particularly that specific for the Fc region, can be minimized by judicious choice of the reagents of the assay. F(ab′)₂ or Fab fragments of murine 4B5 and other reagents such as humanized 4B5 or 4B5, with fewer mouse determinants are appropriate.

After the sample is suitably prepared, it is mixed with a excess 4B5 under conditions that permit formation of a complex between 4B5 and any target antibody that may be present. The amount of complex is then determined, and compared with complexes formed with standard samples containing known amounts of target antibody in the range expected. Complex formation can be observed by any method known in the art such as immunoprecipitation or nephelometry, but it is generally more sensitive to employ a reagent labeled with such labels as radioisotopes such as ¹²⁵I, enzymes such as peroxidase and β-galactosidase, or fluorochromes such as fluorescein.

Pharmaceutical Compositions of the Invention

The present invention encompasses pharmaceutical compositions and immunogenic compositions containing 4B5 either alone or in combination. Such pharmaceutical compositions and vaccines are useful for eliciting an immune response and treating neoplastic diseases, either alone or in conjunction with other forms of therapy, such as chemotherapy or radiotherapy.

The preparation of pharmaceutical compositions that contain 4B5 antibody, or a polynucleotide or a polypeptide derivative thereof as an active ingredient is conducted in accordance with generally accepted procedures for the preparation of pharmaceutical preparations. See, for example, Remington's Pharmaceutical Sciences 18th Edition (1990), E. W. Martin ed., Mack Publishing Co., Pa. Depending on the intended use and mode of administration, it may be desirable to process the active ingredient further in the preparation of pharmaceutical compositions. Appropriate processing may include sterilizing, mixing with appropriate non-toxic and non-interfering components, dividing into dose units, and enclosing in a delivery device.

Liquid pharmaceutically acceptable compositions can, for example, be prepared by dissolving or dispersing a polypeptide embodied herein in a liquid excipient, such as water, saline,, aqueous dextrose, glycerol, or ethanol. The composition can also contain other medicinal agents, pharmaceutical agents, adjuvants, carriers, and auxiliary substances such as wetting or emulsifying agents, and pH buffering agents.

Pharmaceutical compositions of the present invention are administered by a mode appropriate for the form of composition. Typical routes include subcutaneous, intramuscular, intraperitoneal, intradermal, oral, intranasal, and intrapulmonary (i.e., by aerosol). Pharmaceutical compositions of this invention for human use are typically administered by a parenteral route, most typically intracutaneous, subcutaneous, or intramuscular.

Pharmaceutical compositions for oral, intranasal, or topical administration can be supplied in solid, semi-solid or liquid forms, including tablets, capsules, powders, liquids, and suspensions. Compositions for injection can be supplied as liquid solutions or suspensions, as emulsions, or as solid forms suitable for dissolution or suspension in liquid prior to injection. For administration via the respiratory tract, a preferred composition is one that provides a solid, powder, or liquid aerosol when used with an appropriate aerosolizer device. Although not required, pharmaceutical compositions are preferably supplied in unit dosage form suitable for administration of a precise amount Also contemplated by this invention are slow release or sustained release forms, whereby a relatively consistent level of the active compound are provided over an extended period.

Compositions embodied in this invention can be assessed for their ability to recognize specifically a neoplasia. Accordingly, test compounds are prepared as a suitable pharmaceutical composition and administered to test subjects. Initial studies are preferably done in small animals such as mice or rabbits, optionally next in non-human primates and then ultimately in humans. Immunogenicity is preferibly tested in individuals without a previous antibody response. A test composition in an appropriate dose is administered on an appropriate treatment schedule. It may be appropriate to compare different doses and schedules within the predicted range. Such testing is within the skill of one in the art.

Compositions of this invention are particularly suitable for administration to humans with a neoplastic disease. Especially relevant are melanoma, neuroblastoma, glioma, sarcoma, lymphoma, and small cell lung cancer.

The present invention includes methods of eliciting an immune response in a cancer patient that entail administering an effective amount of 4B5 to the individual. In this context, an “effective amount” is an amount sufficient to elicit an immune response, whether humoral and/or cellular. Preferably, the immune response includes the production of anti-GD2.

In a preferred embodiment, 4B5 is used to elicit an immune response and/or for treatment of and/or for palliating advanced cancer. A suitable cancer is one that expresses GD2, expressed on the surface of tumor cells, such as lung cancers and melanoma. As used herein, “advanced” cancers means that there is detectable metastasis, that is, detectable tumor masses at sites other than the primary site of the tumor. Masses are preferably detected by imaging techniques known in the art such as X-ray or CT scan. For eliciting an immune response, palliation, or treatment, an effective amount of 4B5 is administered to an individual with advanced tumor(s). Administration of an effective amount of 4B5 to individuals with advanced cancer may delay or slow the rate of progression of the disease or ameliorate disease, in comparison with other individuals who are not so treated.

It is understood that for some situations involving advanced cancers, the individual receiving 4B5 may be moderately to severely immunocompromised, either due to the nature of previous treatment, the disease itself, or both. Thus, the time required to mount an immune response and/or the number of injections of 4B5 and/or the amount of 4B5 per administration may vary. For example, an individual may require a longer time to elicit an immune response once 4B5 has been administered. In this case, it is recommended that the individual continue to be monitored for an immune response, even if no initial (i.e., within the first month) no immune response has been detected. As another example, an individual may require more than the average number of injections to elicit an immune response.

The effective amount of 4B5 antigen binding fragments to be administered will depend upon several factors, such as the route of administration, the condition of the individual, and the desired objective. The term “therapeutically effective” means that the amount of antigen binding fragment used is of sufficient quantity to ameliorate the cancer. “Ameliorate” denotes a lessening of the detrimental effect of the cancer on the individual. Typically, if administered directly, the amount per administration is about 10 μg to 20 mg, preferably 250 μg to 10 mg, more preferably 300 μg to 5 mg, even more preferably 500 μg to 2.5 mg. Administrations are typically conducted on a weekly or biweekly basis until a desired, measurable parameter is detected, such as diminution of disease symptoms. Administration can then be continued on a less frequent basis, such as biweekly or monthly, as appropriate.

4B5 is typically administered bi-weekly for four injections, followed by monthly injections as required. Timing of subsequent injections (i.e., a maintenance dose) will depend, inter alia, upon the condition and response of the individual being treated. Ab3 levels can be monitored, for example, preferably by the diagnostic methods described herein, to determine when maintenance (booster) administrations should be given, which would typically be about every three months.

The dosage ranges for the administration of 4B5 are those large enough to produce the desired effect in which the symptoms of the malignant disease are ameliorated without causing undue side effects such as unwanted cross-reactions, anaphylactic reactions, and the like. Generally, the dosage will vary with the patient's age, condition, sex and extent of the disease and can be determined by one of skill in the art. The dosage can be adjusted by the individual physician in the event of any complication. Dosage can vary from about 0.1 mg/kg to about 2000 mg/kg, preferably about 0.1 mg/kg to about 500 mg/kg, in one or more dose administrations daily, for one or several days. Generally, when 4B5 are administered conjugated with therapeutic agents, lower dosages, comparable to those used for in vivo immunodiagnostic imaging, can be used.

Another method of administration is intralesionally, for instance by direct injection directly into the tumor. Intralesional administration of various forms of immunotherapy to cancer patients does not cause the toxicity seen with systemic administration of inununologic agents. Fletcher et al. (1987) Lymphokine Res. 6:45; Rabinowich et al. (1987) Cancer Res. 47:173; Rosenberg et al. (1989) Science 233:1318; and Pizz et al. (1984) Int. J. Cancer 34:359.

One possible indication of effectiveness of administration of 4B5, whether for eliciting an immune response and/or treatment, or whether administration of 4B5 is indicated, is the density of GD2 on the tumor cells. This density can vary widely from individual to individual, and may vary over the course of administration of 4B5 and/or over the course of the disease. As used herein, “density” of GD2 can refer to either or both of the following: (a) the number of cells per total cells in a given biological sample that have GD2 on their surface; (b) the amount of GD2 on the surface of each cell. Density (a) is calculated by noting the number of cells in a sample that are stained or otherwise indicate that GD2 is present divided by the total number of cells. This density would be preferably greater than about 20%, more preferably greater than about 30%, more preferably greater than about 50%, even more preferably greater than about 70%, even more preferably greater than about 80%, most preferably greater than about 90%. Thus, the invention includes administration of 4B5 to an individual having density of GD2 greater than about 20%, preferably greater than 30%, more preferably greater than 70%, even more preferably greater than about 80%, most preferably greater than about 90%.

Density (b) is indicated by the relative intensity of staining (or intensity of any measurement indicating the presence of GD2) of cells in a sample from one individual relative to, for example, a sample from another individual. For this density, one skilled in the art can make an empirical determination of density. Density (b) is relative to normal tissues (i.e., cells lacking GD2, or unaffected cells), so preferred ranges may be about 1.5 fold, preferably about 3 fold, more preferably about 10 fold higher expression over unaffected cells, as detected by immunohistochemical staining density. Unaffected cells could also be from the same individual.

This is not to say that individuals with lower densities, for example, less than about 50% are not indicated for administration of 4B5. While not wishing to be bound by a single theory, it is possible that administration of 4B5 could elicit a series of immuno-reactions that result in a more general response that is effective against a GD2-associated tumor, such as a cytotoxic T cell response. A lower density, however, may indicate that additional therapies are desirable.

It is understood that density can also be used as an indicator of extent of disease and response to administration of 4B5. For example, a sample taken from an individual at the onset of 4B5 administration may exhibit about 80% density (i.e., about 80% of the cells exhibit GD2). After receiving 4B5, a sample taken from the same location may exhibit only about 50% density, indicating that GD2-expressing cells are being destroyed. Similarly, if the intensity of staining of a sample from an individual receiving 4B5 diminishes upon receiving 4B5, this indicates that GD2-bearing tumor cells are being destroyed.

For purposes of raising an immune response or providing treatment to individuals with advanced GD2-associated tumors, 4B5 is administered parenterally, preferably intacutaneously. Other routes of administration include, but are not limited to, intramuscular, subcutaneous and intradermal. 4B5 can also be administered indirectly, by treatment of cultured cells followed by introduction of these cultured cells into an individual.

Preferably, 4B5 is administered with a pharmaceutically acceptable excipient. A pharmaceutically acceptable excipient is a relatively inert substance that facilitates administration of a pharmacologically effective substance. For example, an excipient can give form or consistency to the vaccine composition, or act as a diluent. Suitable excipients include but are not limited to stabilizing agents, wetting and emulsifying agents, salts for varying osmolarity, encapsulating agents, buffers, and skin penetration enhancers. Examples of pharmaceutically acceptable excipients are described in Remington's Pharmaceutical Sciences (Alfonso R. Gennaro, ed., 18th edition, 1990).

Preferably, 4B5 is used with an adjuvant that enhances presentation of 4B5 or otherwise enhances the immune response against 4B5. Suitable adjuvants include aluminum hydroxide, alum, QS-21 (U.S. Pat. No. 5,057,540), DHEA (U.S. Pat. Nos. 5,407,684 and 5,077,284) including its precursors and modified forms, (e.g., DHEA-S, the sulfonated form of DHEA), β-2 microglobulin (WO 91/16924), muramyl dipeptides, muramyl tripeptides (U.S. Pat. No. 5,171,568) and monophosphoryl lipid A (U.S. Pat. No. 4,436,728; WO 92/16231) and its derivatives, e.g., Detox™, and BCG (U.S. Pat. No. 4,726,947). Other suitable adjuvants include, but are not limited to, aluminum salts, squalene mixtures (SAF-1), muramyl peptide, saponin derivatives, mycobacterium wall preparations, mycolic acid derivatives, nonionic block copolymer surfactants, Quil A, cholera toxin B subunit, polyphosphazene and derivatives, and immunostimulating complexes (ISCOMs) such as those described by Takahashi et al. (1990) Nature 344:873-875. For veterinary use and for production of antibodies in animals, mitogenic components of Freund's adjuvant can be used.

The choice of an adjuvant will depend in part on the stability of the vaccine in the presence of the adjuvant, the route of administration, and the regulatory acceptability of the adjuvant, particularly when intended for human use. For instance, alum is approved by the United States Food and Drug Administration (FDA) for use as an adjuvant in humans. The 4B5 can be administered in a precipitated form; for example, alum-precipitated 4B5 can be used. If QS-21 is used, preferably 100 μg is used for each dose which preferably is administered subcutaneously within about 30 minutes of mixing with 4B5 (with care taken to mix gently). If DetoxTM is used, preferably 0.12 ml is used for each dose, which is preferably administered subcutaneously within about 30 minutes of mixing with 4B5. Manufacturers of adjuvants generally provide recommendations regarding amounts, volume, preparation, and route(s) of administration.

Alternatively, 4B5 can be encapsulated, for example, in liposomes. Liposomes suitable for packaging polypeptides for delivery to cells are known in the art.

4B5 can be heat treated before administration, and the heat treatment can be in the presence of adjuvant, for example, alum. For instance, 4B5 can be heated at about 40 to 60° C., preferably 45° C. to 55° C., for a period of about 5 minutes to 2 hours, preferably 15 minutes to 1 hour. Heat treatment is more preferably at 45° C. for 30 minutes in a sterile vial, in a water bath. The heat treatment can occur anytime before administration. Preferably, heat treatment is within 7 days of administration. Other heat treatment procedures can be used, as long as the desired activity of 4B5 is not significantly compromised.

For the purpose of raising an immune response, 4B5 can be administered in an unmodified form. 4B5 can be modified to improve its immunogenicity. Methods of improving immunogenicity include, inter alia, crosslinking with agents such as gluteraldehyde or bifunctional couplers, or attachment to a polyvalent platform molecule. Immunogenicity may also be improved by coupling to a protein carrier, particularly one that comprises T cell epitopes.

4B5 can be used alone or in conjunction with other agents which promote the desired activity/objective. In this context, an “agent” can be any of a variety of substances. Further, “in conjunction with” means that the agent can be used concomitantly, before, or after 4B5. A desired activity is any activity that facilitates, enhances, promotes, or modulates the desired objective in using 4B5. Agents wnich can be used include, but are not limited to, cytokines, lymphokines, adjuvants, and drugs. Agents also include substances which facilitate delivery of 4B5, such as liposomes, or substances which promote delivery of 4B5 to a particular target, for example, a cellular receptor. For example, 4B5 can be administered with a cytokine such as GM-CSF.

In order to determine the effect of administration with 4B5, an individual can be monitored for either an antibody (humoral) or cellular immune response against GD2, or a combination thereof.

To determine the level of GD2 antibody (Ab3) in a biological sample, for example, serum or plasma is obtained from the individual. Optionally, the sample can be enriched for immunoglobulin before the assay is conducted, although this is not usually required. If a mouse immunoglobulin is to be used as an assay reagent, the sample is preferably pretreated to remove anti-mouse immunoglobulin activity. This can be performed, for example, by depletion on a mouse immunoglobulin column, or by mixing non-specific mouse immunoglobulin into the sample and removing any immunoprecipitate formed.

To conduct the assay, anti-GD2 that may be in the sample is contacted with a non-limiting amount of an antigenic equivalent of GD2. This may be isolated GD2, nitrocellulose with GD2 affixed by direct blotting or by transfer from a polyacrylamide gel, cells expressing GD2, membrane preparations from such cells, or fixed tissue sections containing GD2. Alternatively, an anti-idiotype, particularly 4B5 may be used.

Once the immune complex has formed, it is generally separated from uncomplexed GD2 analog, and the amount of complex present is determined. The complex can be separated, for example, by centrifugation to collect cells or an immunoprecipitate, or capture by a solid phase. The amount of complex present can be measured by providing the GD2 analog with a label either directly, or by incubating with a secondary reagent. Alternatively, a competition assay may be performed, in which the sample is first incubated with the GD2 analog, and then a non-limiting amount of a labeled anti-GD2 reagent is added which competes with the anti-GD2 which may be present in the sample. Suitable labels include radiolabels, enzyme labels, fluorescent labels, and chemiluminescent labels. A standard curve is constructed using solutions known to contain no anti-GD2, and solutions with various relative concentrations of anti-GD2, in place of the sample. The sample containing the unknown amount of anti-GD2 is generally assayed in parallel, and the relative amount of anti-GD2 contained therein is determined by comparison with the standard curve. Preferred assays for determining anti-GD2 levels using 4B5 antibody are described in more detail in a following section.

The isotype of the anti-GD2 antibody can be determined by including in the immunoassay an isotype-specific reagent, either at the separation or the labeling stage. For example, anti-human IgG may be used to separate or detect antibody of the IgG class present in a clinical sample of human origin. Presence of specific anti-GD2 of the IgG class generally indicates a memory response. Presence of anti-GD2 of the IgM class generally indicates ongoing immunostimulation, such as may be due to the presence of an GD2 expressing tumor, or ongoing treatment with 4B5.

If desired, anti-GD2 antibody detected in a biological sample can be further characterized; for example, by competition with anti-GD2 (Ab1) to determine whether they are specific for related epitopes on GD2. Competition assays between Ab1 and Ab3 are known in the art and not described in detail herein.

Anti-GD2 antibody can also be tested to determine whether it is cytotoxic. Complement mediated cytotoxicity (CMC) is determined, for example, by using GD2-expressing target cells labeled with ⁵¹ Cr. Labeling can be accomplished by incubating about 10⁶ cells with ˜200 μCi Na₂⁵¹CrO₄ for 60 minutes at 37° C., followed by washing. The assay is conducted by incubating the antibody (or clinical sample containing the antibody) with the target cells. The opsonized cells are then washed and incubated with a source of complement; for example, guinea pig serum pre-adsorbed to remove intrinsic antibody activity. After a suitable incubation period at 37° C., release of ⁵¹Cr into the medium is determined and compared with that from unopsonized control cells. Release of ⁵¹Cr correlates with CMC activity.

Another way of characterizing the anti-GD2 antibody is by testing its ability to participate in an ADCC response (Cheresh et al. (1986), Cancer Res. 46:5112). Radiolabeled GD2-expressing target cells are incubated with the anti-GD2 (in the form of heat-inactivated serum), and effector cells. Normal human peripheral blood mononuclear cells (PBMC) are suitable effector cells, and preferably are used at an effector:target ratio of about 100. After approximately 4 hours at 37° C., the proportion of released ⁵¹Cr is determined as a measure of ADCC activity.

The cellular immune response in a subject being administered 4B5 may be quantified by conducting standard functional assays for specific T cell activity.

One type of assay measures T cell proliferation. In this test, peripheral blood mononuclear cells (PBMC) are obtained from a whole blood sample collected from the treated subject. For experimental animals, spleen cells can also be used. T cells can be enriched, for example, by centrifugation on a gradient such as Ficoll(™). The cells are then cultured in the presence of GD2 or (more usually) irradiated GD2 expressing cells at various concentrations. Preferably, the stimulator cells are autologous with the responder cells, particularly in terms of histocompatibility Class II antigens.

Another type of assay measures T cell cytotoxicity. In this test, an enriched T-cell population is used to effect lysis of ⁵¹Cr-labeled GD2 expression target cells, prepared as described above. Preferably, the effector cells are autologous with the target cells, particularly in terms of histocompatibility Class I antigens. The T cell population can optionally be pre-stitnulated with GD2 or a relevant cell line. The T cells are then combined at various ratios with about 10⁴ labeled target cells; for example, in wells of a microtiter plate. The plate is optionally centrifuged to initiate cell contact, and the cells are cultured together for 4-16 hours at 37° C. The percent specific release of ⁵¹Cr into the medium is measured in comparison with labeled targets cultured alone (negative control) and targets lysed with a detergent such as 0.1% Triton (™) X-100 (positive control).

Other relevant measurements to determine the effect of 4B5 administration include clinical tests as may be appropriate in determining the progression of cancer of the suspected type. Such tests include but are not limited to, inflammatory indicators, mammography, and radioscintigraphy.

Another way that 4B5 can be used is to assay for the presence of an antibody or other immune component that binds to 4B5, or to GD2. Such components may be present following therapeutic administration of 4B5, or may spontaneously arise due to the presence of an GD2-expressing tumor in an immunocompetent host. Assays can be conducted on biological samples, usually clinical samples.

In one embodiment of this invention, 4B5 is used to detect the presence of an anti-GD2, particularly anti4B5 idiotype, that may be present in a biological sample. The sample is suitably prepared before conducting the assay, optionally by enriching for antibody activity. If the biological sample is suspected of containing antibody activity against non-idiotypic regions of 4B5 particularly anti-mouse immunoglobulin), it is preferable to remove them or conduct the assay so as to avoid their detection. Anti-mouse immunoglobulin antibody can be removed from a sample, for example, by precipitation with normal mouse IgG or adsorption with a mouse Ig adsorbant. Binding of anti-mouse immunoglobulin antibody, particularly that specific for the Fc region, can be minimized by judicious choice of the reagents of the assay. F(ab′)₂ or Fab fragments of 4B5 and other mouse immunoglobulin reagents are especially appropriate.

After the sample is suitably prepared, it is mixed with a excess finctional equivalent of 4B5 under conditions that permit formation of a complex between 4B5 and any anti-GD2 that may be present. The amount of complex is then determined, and compared with complexes formed with standard samples containing known amounts of anti-GD2 in the range expected. Complex formation can be observed by immunoprecipitation or nephelometry, but it is generally more sensitive to employ a reagent labeled with such labels as radioisotopes like ¹²⁵I, enzymes like peroxidase and β-galactosidase, or fluorochromes like fluorescein.

Antibody assays can be conducted in fluid phase. For example, anti-GD2 may be mixed with labeled 4B5. Alternatively, the anti-GD2 in the sample can be used to compete with a labeled anti-GD2 for binding sites on 4B5. Generally, bound and unbound label is separated to quantitate the percent bound. Suitable separation methods include gel filtration chromatography, and precipitation with antibody against immunoglobulin of the species from which the sample is obtained, optionally in the presence of polyethylene glycol. Alternatively, the proportion of bound and unbound label may be determined in situ, for example, using fluorescence/quench labeling pairs or enzyme/inhibitor labeling pairs. See, e.g., U.S. Pat. No. 3,996,345 (Ullman et al.).

As described above, it is generally more convenient to conduct a capture assay using a reagent linked to a solid phase, such as a polyethylene test tube, microtiter plate well, or magnetic bead. In a competition-type capture assay, unlabeled anti-GD2 in the sample competes with a labeled anti-GD2 reagent for binding to 4B5. The 4B5 may be attached directly to the solid support, or captured later, for example, using an anti-4B5. In this assay, the amount of label associated with the solid phase is inversely related to the amount of anti-GD2 in the sample.

In the sandwich-type capture assay, anti-GD2 is captured by 4B5 attached directly or through a secondary reagent to a solid phase. After washing, the anti-GD2 is detected using anti-immunoglobulin of the appropriate species, or a second 4B5 antibody, to which a label is directly or indirectly attached. Alternatively, the anti-immunoglobulin may be attached to the solid phase and labeled 4B5 is used to complete the sandwich. If the anti-immunoglobulin used is isotype-specific, then the class of the antibody can also be determined. In this type of assay, the amount of label associated with the solid phase correlates positively with the amount of anti-GD2 in the sample.

Other methods of measuring specific antibody are known in the art, and can be adapted to measure anti-GD2 by using 4B5 as the target antigen. All such adapted methods are embodied in this invention.

4B5 can also be used to measure the level of cellular anti-GD2 activity, particularly anti-4B5 idiotype. In a preferred example, 4B5 is used to identify anti-GD2 T cells, defined for this purpose as lymphocytes expressing a T cell receptor that binds the 4B5 idiotype. 4B5 may be labeled and contacted with a population of cells suspected of containing anti-GD2 T cells. Alternatively, unlabeled 4B5 may be mixed with the cells, and followed with a labeled secondary reagent such as labeled anti-mouse immunoglobulin or protein A. Suitable labels for this purpose include radiolabels and fluorescent labels. The use of fluorescent labels would also allow anti-GD2 cells to be separated from non-specific cells in a fluorescence-activated cell sorter.

The invention also encompasses methods using 4B5 to remove a label, for example radioactivity, from an individual who has received a labeled anti-GD2 antibody (AbI), for example, for radioscintilligraphy or radiotherapy. One problem common to use of antibody targeted radionuclides (i.e., radioimmunotherapy) has been the presence of excess Ab1 in the system which limits the dosage of radiolabeled antibody for treatment. Further, effective imaging using radiolabeled antibodies is hampered due to excess circulating radiolabeled antibody, which often takes several days to clear circulation and tissues. In these methods of the present invention, 4B5 is administered to the individual at a specified time after administration of the labeled anti-GD2. The intention is for the 4B5 to complex with anti-GD2 at sites other than the tumor, such as in the circulation and interstitial spaces, and thereby promote its clearance. As a result, the level of labeled moiety (such as radioisotope) in unaffected tissues is reduced, and the image of the tumor (in comparison to neighboring tissues) is enhanced. Similarly, when radionuclides are given to subjects for irradiation of a tumor site, it is desirable to reduce collateral exposure of unaffected tissue. This invention thus includes methods of treatment in which a radiolabeled anti-GD2 antibody is administered in a therapeutic dose, and followed by a molar excess of 4B5.

In either of these applications, an amount of 4B5 is chosen that is in sufficient molar excess over the labeled anti-GD2 to locate and bind any anti-GD2 that is not localized at the tumor site. The timing of administration and amount of 4B5 will depend upon the nature of the radiolabeled antibody, the type of radioisotope used and the condition of the individual. Preferably, the molar ratio of 4B5 to the anti-GD2 antibody is at least about 5: 1, more preferably about 25:1 to 200:1. Preferably, 4B5 is administered 5 to 24 hours after the individual has received the anti-GD2 antibody.

The invention also includes methods of detecting the presence of an anti-GD2 antibody bound to a tumor cell comprising the steps of treating the tumor cell with 4B5 for a sufficient time to allow binding to the anti-GD2 antibody, and detecting the presence of any complex formed. The intention is for the 4B5 to detect anti-GD2 that has pre-attached to the tumor cell; or alternatively, to promote the binding of anti-GD2 to the tumor cell by forming a polyvalent anti-GD2/4B5 immune complex. In the former instance, the 4B5 is provided with a detectable label or a means by which a label can be attached. In the latter instance, either the anti-GD2 or the 4B5 is provided with a label.

This strategy can be used, for example, to identify an GD2 antigen-bearing cell in a isolated cell suspension. The cells are incubated sequentially or simultaneously with anti-GD2 and 4B5, washed, and then the labeled cells are detected. Preferred labels for this embodiment include fluorescent labels, such as fluorescein, rhodamine, and Texas red. Optionally, labeled cells can be separated from unlabeled cells; for example, by sorting in a fluorescence-activated cell sorter or by affinity separation, using any of the solid phase positive or negative immunoselection techniques known in the art.

The strategy can also be used, for example, to detect or image tumors in an affected subject. The anti-GD2 and 4B5 are administered (usually sequentially) into the subject and allowed to accumulate at the tumor site. Suitable labels include radiolabels such as ¹¹¹In, 131I and ^{99m}Tc. The tumor is then detected or visualized using standard techniques of radioscintigraphy.

The various compositions of this invention can be used alone, or in conjunction with other active agents that promote the desired objective, or provide a desirable adjunct therapy. Suitable active agents include the anti-neoplastic drugs and bioresponse modifiers described above and effector cells such as those described by Douillard et al. (1986) Hybridomas (Supp. 1:5139).

Using 4B5, it is possible to design combination therapies. It may be desirable to administer a therapeutic agent, or agents, prior to the administration of 4B5 in combination with effector cells and the same, or different, therapeutic agent or agents. For example, patients can be treated by first administering IFN-γ and interleukin-2 (Il-2) daily for 3 to 5 days, and on day 5 administering 4B5 in combination with effector cells, IFN-γ, and Il-2.

Suitable subjects for treatment with 4B5 or combination therapies including 4B5, include those who are suspected of being at risk of a pathological effect of any neoplasia, particularly carcinoma, are suitable for treatment with the pharmaceutical compositions of this invention. Those with a history of cancer are especially suitable. Suitable human subjects for therapy comprise two groups, which can be distinguished by clinical criteria. Patients with “advanced disease” or “high tumor burden” are those who bear a clinically measurable tumor. A clinically measurable tumor is one that can be detected on the basis of tumor mass (e.g., by palpation, CAT scan, or X-Ray; positive biochemical or histopathological markers on their own may be insufficient to identify this population). A pharmaceutical composition embodied in this invention is administered to these patients to elicit an anti-tumor response, with the objective of palliating their condition. Ideally, reduction in tumor mass occurs as a result, but any clinical improvement constitutes a benefit. Clinical improvement includes decreased risk or rate of progression or reduction in pathological consequences of the tumor.

A second group of suitable subjects is known in the art as the “adjuvant group”. These are individuals who have had a history of cancer, but have been responsive to another mode of therapy. The prior therapy may have included, but is not restricted to, surgical resection, radiotherapy, and traditional chemotherapy. As a result, these individuals have no clinically measurable tumor. However, they are suspected of being at risk for progression of the disease, either near the original tumor site, or by metastases.

This group can be further subdivided into high-risk and low-risk individuals. The subdivision is made on the basis of features observed before or after the initial treatment. These features are known in the clinical arts, and are suitably defined for each different cancer. Features typical of high risk subgroups are those in which the tumor has invaded neighboring tissues, or who show involvement of lymph nodes.

Another suitable group of subjects is those with a genetic predisposition to cancer but who have not yet evidenced clinical signs of cancer. For instance, women testing positive for a genetic mutation associated with breast cancer, but still of childbearing age, may wish to receive 4B5 treatment prophylactically to prevent the occurrence of cancer until it is suitable to perform preventive surgery.

A pharmaceutical composition embodied in this invention is administered to patients in the adjuvant group, or in either of these subgroups, in order to elicit an anticancer response. Ideally, the composition delays recurrence of the cancer, or even better, reduces the risk of recurrence (i.e., improves the cure rate). Such parameters may be determined in comparison with other patient populations and other modes of therapy.

Of course, crossovers between these two patient groups occur, and the pharmaceutical compositions of this invention can be administered at any time that is appropriate. For example, 4B5 therapy can be conducted before or during traditional therapy of a patient with high tumor burden, and continued after the tumor becomes clinically undetectable. 4B5 therapy can be continued in a patient who initially fell in the adjuvant group, but is showing signs of recurrence. The attending physician has the discretion to determine how or when the compositions of this invention are to be used.

In another embodiment, 4B5 polypeptides can be conjugated with carrier. In instances where the 4B5 polypeptide is correctly configured so as to provide a binding site, but is too small to be immunogenic, the polypeptide may be linked to a suitable carrier. A number of techniques for obtaining such linkage are known in the art and need not be described in detail herein. Any carrier can be used which does not itself induce the production of antibodies harmful to the host. Suitable carriers are typically large, slowly metabolized macromolecules such as proteins; polysaccharides, such as latex functionalized Sepharose, agarose, cellulose, cellulose beads and the like; polymeric amino acids, such as polyglutamic acid, polylysine, and the like; amino acid copolymers; and inactive virus particles or attenuated bacteria, such as Salmonella. Especially useful protein substrates are serum albumins, keyhole limpet hemacyanin, immunoglobulin molecules, thyroglobulin, ovalbumin, tetanus toxoid, and other proteins well known to those of skill in the art. As is evident to one skilled in the art, the above-described recombinant forms of 4B5 polypeptides and 4B5, such as fusion proteins, can in turn be fused with other amino acid sequences. For instance, a 4B5 scfv can be fused to a cytokine, such as IL-2.

The ability of a 4B5 polypeptide to bind AbI and/or Ab3 can be assessed several ways. In one test, binding of the 4B5 polypeptide(s) to Ab1 can be tested directly, for example, by radioimmunoassay (RIA), for example, by reacting radiolabeled 4B5 polypeptide with Ab1 or Ab3 coated on microtiter plates.

In another procedure, binding to Ab1 or Ab3 is determined by competitive immunoassay, In one variation of this procedure, binding of labeled 4B5 polypeptide(s) or functional equivalent fragments to Ab1 is measured in the presence of different Ab1, other Ab2s, 4B5 or analogs thereof, other 4B5 polypeptide(s), GD2 or extracts containing GD2, or other proteins. Percent inhibition is calculated according to the following formula:

$$\% \text{inhibition} = [ 1 - ( \frac{{R}_{T} - {R}_{C}}{{R}_{MAX} - {R}_{C}} ) ] \times 100 \%$$

In another variation, the test fragment with putative 4B5 activity is tested for its ability to interfere with the binding between Ab1 and Ab2, or Ab1 and GD2. This test may be more sensitive in some applications, because lower affinity interaction between 4B5 and Ab1 may be too weak to form a stable bond, but be adequate to interfere with the binding of another ligand-receptor pair when present at sufficient concentration. The GD2 can be provided as purified antigen or GD2-expressing cells. The assay can be conducted by labeling either the Ab1 or the GD2 or Ab2, and optionally immobilizing the other member of the ligand-receptor pair on a solid support for ease of separation. The test fragment is incubated with the labeled reagent, and then the mixture is presented to the immobilized target or test cell to determine if the test fragment is able to inhibit binding. Degree of inhibition correlates with 4B5 activity.

Various examples of competition assays are known in the art and include the following. One test that indicates 4B5 polypeptide activity is to measure the binding of radiolabeled Ab 1 to semipurified or purified GD2 in the presence of varying amounts of 4B5 polypeptide(s). The Ab1 -GD2 mixture is then added to plates coated with 4B5 polypeptide(s) and binding is compared with binding of labeled Ab1 alone. Preferably, this test is performed with nonsaturating amounts of labeled Ab1 to detect changes in binding with small amounts of competitive GD2. In another competition assay, GD2 positive target cells are grown in 96-well tissue culture plates as a confluent monolayer. Binding of radiolabeled Ab1 in the absence and presence of 4B5 polypeptides is determined. The degree of inhibition can be compared with that of intact 4B5 or other 4B5 polypeptides.

A 4B5 polypeptide is considered to bind Ab1 if there is inhibition when compared to a negative control, such as an unrelated anti-idiotype antibody which does not bind to Ab1.

With all of the above-described assays, it is clear to one of skill in the art that the labeled molecule can be labeled in various ways, such as with radioisotopes (i.e., ¹²⁵I) and non-radioactive labels, such as biotinylated molecules, and molecules for enzymatic detection, fluorescent labels and chemiluminescent labels.

The above discussed tests can also be used to compare characteristics of various 4B5 polypeptide fragments. For example, competitive assays can be conducted in which a first 4B5 polypeptide competes for binding to Ab1 in the presence of varying amounts of a second 4B5 polypeptide. Such tests can indicate relative degrees of binding affinities or other characteristics.

Another way of characterizing 4B5 polypeptides is testing their ability to generate an immune response. As used herein, “immune response” indicates either a humoral response, a cellular response, or both. As used herein, the “ability to elicit an immune response” pertains to any individual, including human.

The ability of a 4B5 polypeptide to generate a humoral response can be determined by testing for the presence of an antibody that binds to the 4B5 polypeptide(s) after administration of the 4B5 polypeptide(s). It is understood that this antibody (Ab3) was not present, or was present in lower amounts, before administration of the 4B5 polypeptide(s). Immunogenicity is preferably tested in individuals without a previous anti-4B5 response. Examples of suitable individuals include, but are not limited to, mice, rabbits, monkeys and humans. For this test, an individual is administered a 4B5 polypeptide(s). The amount per administration and number of administrations will vary, depending on the individual. The requisite amount can be determined readily by empirical observations.

Presence of an Ab3 can be determined by first pre-incubating sera with autologous immunoglobulin to block antibodies against isotypic and allotypic determinants and then testing sera for binding to GD2 and/or the 4B5 polypeptide(s), for example, using ELISA or RIA. For instance, different dilutions of pre-reacted sera are reacted with 4B5 coated on microtiter plates. An unrelated Ab2 serves as a control. After washing, the Ab3-4B5 complex is labeled using, for example, ¹²⁵I-labeled 4B5 in a homogeneous sandwich assay. Results from this assay are compared to those obtained before administration of the 4B5 polypeptide. Alternatively, binding to GD2 positive cells, can be tested using immune flow cytometry.

Binding of Ab3 to GD2 can also be determined by immunoprecipitation or immunoreactivity with GD2-positive tissue samples, or dot blot analysis. In one method of dot blot analysis, a semi-purified extract of GD2 is directly blotted to a nitrocellulose filter. The filter is then incubated with sera containing Ab3, and the reaction developed by enzyme-conjugated anti-immunoglobulin. If the Ab3 binds to GD2, a positive blot should appear. For testing with tissue samples, an immunoperoxidase assay can be used.

If desired, Ab3 elicited by 4B5 polypeptide(s) can be further characterized. For example, competition assays can be performed to determine whether Ab3 share Ab1 idiotopes. In this test, serum from an individual immunized with a 4B5 polypeptide is tested for inhibition of binding of labeled 4B5 polypeptide (or intact 4B5) to Ab 1. Inhibition indicates that Ab3 and Ab1 contain at least similar binding determinants. Similarly, competition of Ab3 with Ab1 for binding to GD2 (whether partially purified, purified, or on the surface of a GD2-positive cell) can be tested by coincubating a fixed amount of labeled Ab1 with different dilutions of Ab3 containing sera or Ab1 preparation and GD2 As is evident to one of skill in the art, the Ab3 can be used in turn to characterize 4B5 polypeptides, using the assays described above.

Another way of characterizing a 4B5 polypeptide is by testing its ability to elicit an antibody that is cytoxic. Methods for this determination are described above. For determination of complement mediated cytotoxicity (CMC), SKBR3 (target) cells (i.e., cells that express GD2) are labeled with ⁵¹Cr. Labeling can be accomplished by incubating about 10⁶ cells with approximately 200 μCi Na₂SO₄ for 60 minutes at 37° C., followed by washing. The assay is conducted by adding and incubating serum suspected of containing antibody. Guinea pig serum pre-adsorbed with LS174-T cells (or other source of complement) is then added. After a suitable incubation period at 37° C., extent of ⁵¹Cr release is then measured and compared with that of unopsonized control cells. Release of ⁵¹Cr correlates with CMC activity. Herlyn et al. (1981) Int. J. Cancer 27:769.

Another way of characterizing a 4B5 polypeptide is by testing its ability to elicit an anti-GD2 antibody that participates in an ADCC response. Cheresh et al. (1986) Cancer Research 46:5112-5118. In this assay, cultured or GD2-expressing cells are labeled with ⁵¹Cr and are used as target cells. Normal human peripheral blood mononuclear cells (PBMC) are used as effector cells. Preferably, the ADCC assay is conducted in the presence of heat-inactivated serum with an effector to target cell ratio of 100:1 for 4 hours, although other suitable conditions may be used. The amount of ⁵¹Cr released is then measured.

The 4B5 polypeptides of this invention can also be characterized by their ability to elicit a cellular response. As used herein, a “cellular response” is a response that involves T cells, and can be observed in vitro or in vivo.

One way of detecting a cellular immune response is by assaying for T cell proliferative activity. In this test, cellular immune response is measured by proliferation of peripheral blood mononuclear cells (PBMs) incubated with 4B5 polypeptide(s). Peripheral blood mononuclear cells are isolated from blood after a requisite number of administrations of 4B5 polypeptide(s) and are incubated with varying concentrations of 4B5 polypeptide(s). If mice are used, T cells are obtained from spleen. T cells can be enriched, for example, by centrifugation on a gradient such as FicollTM. A non-specific mitogen such as PHA serves as a positive control; incubation with an unrelated anti-idiotype antibody serves as a negative control. Preferably, the stimulator cells are autologous with the responder cells, particularly in terms of histocompatibility Class II antigens. After incubation of the PBMs for an appropriate number of days to allow proliferation. [³H]thymidine incorporation is measured. In many instances a suitable time is five days. If desired, determination of which subset of T cells are proliferating can be performed using flow cytometry. Optionally, splenic T cells can be pre-depleted of either CD4⁺ or CD8⁺ cells before the proliferation assay by incubation with monoclonal antibodies such as RL. 172 (anti-CD4⁺) or mAb. 168 (anti-CD8⁺) and complement.

Another way of detecting a cellular immune response is to test for T cell cytotoxicity (CTL) activity. In this test, T lymphocytes (i.e., an enriched T cell population) are isolated (typically from spleen cells) for use as targets in a standard ⁵¹Cr release assay. Kantor et al. (1992) J. Natl. Cancer Inst. 84:1084-1091. An example of a ⁵¹Cr release assay is described above.

Another way of characterizing 4B5 polypeptides is testing their ability to ameliorate, delay the progression of and/or reduce the extent of GD2-associated tumors. Such tests may include inflammatory indicators, radioscintigraphy, or measurement of circulating GD2 levels (such assays are available commercially).

In summary, 4B5 polypeptides have a number of uses. 4B5 polypeptides can be used to induce an immune response in an individual, preferably an anti-GD2 response. They can also be used to detect and monitor levels of Ab3, or to purify Ab3. 4B5 polypeptides are also useful for treatment of GD2-associated disease, for example, certain lung cancers and melanoma.

Thus, the present invention includes methods of inducing an immune response in an individual comprising administering a 4B5 polypeptide in an amount effective to induce an immune response. Preferably, the individual has GD2-associated tumors. In this context, an “effective amount” is an amount sufficient to elicit a measurable immune response, whether humoral and/or cellular. An effective amount can be administered in one or more administrations.

The invention also encompasses methods of detecting an antibody that binds to 4B5 (i.e., Ab3 and/or Ab1) in abiological sample. These methods are applicable in e clinical setting, for example, for monitoring Ab1 or Ab3 levels in an individual, as well as an industrial setting, in which commercial production of Ab3 is desired. These methods entail contacting the Ab3 and/or Ab1 in the sample with a 4B5 polypeptide under conditions suitable to allow the formation of a stable complex between Ab3 and/or Ab1 and the 4B5 polypeptide, and detecting a stable complex formed, if any. A number of immunoassay methods are known in the art and have been described herein. For further illustration, a test sample potentially containing Ab3 and/or Ab1 can be mixed with a pre-determined non-limiting amount of the 4B5 polypeptide which is typically detectably labeled (such as with a radioisotope or enzyme). In a liquid phase assay, unreacted reagents are removed by a separation technique, such as filtration or chromatography. In these immunoassay techniques, the amount of label associated with the complex positively correlates with the amount of Ab3 and/or Ab1 present in the sample. Similar assays can be designed in which Ab3 and/or Ab1 in the test sample competes with labeled antibody for binding to a limiting amount of the 4B5 polypeptide. Here, the amount of label negatively correlates with the amount of Ab3 and/or Ab1 in the sample. Suitable samples in which to measure Ab3 and/or Ab1 levels are biological samples, including serum or plasma, preferably serum. Other samples include tissue samples.

Further, the invention also includes methods of purifying Ab3 (or Ab1), comprising contacting a biological sample containing Ab3 (and/or Ab1) with a 4B5 polypeptide, and obtaining a complex formed thereby, if any. Typically, the 4B5 polypeptide(s) is coupled to an affinity matrix for affinity column purification. Such methods are routine in the art and need not be described in detail herein.

Kits comprising 4B5 polynucleotides and/or encoded 4B5 The present invention encompasses kits containing 4B5 polynucleotides and 4B5, preferably diagnostic kits. Diagnostic procedures using 4B5 polynucleotides and 4B5 can be performed by diagnostic laboratories, experimental laboratories, practitioners, or private individuals. The clinical sample is optionally pre-treated for enrichment of the target being tested for. The user then applies a reagent contained in the kit in order to detect the changed level or alteration in the diagnostic component.

Kits embodied by this invention include those that allow someone to detect the presence of 4B5 polynucleotides and/or anti-GD2 antibodies (both Ab1 and Ab3). Optionally, reagents such as 4B5 contained in the kits may be conjugated with a label to permit detection of any complex formed with the target in the sample. In another option, a second reagent is provided that is capable of combining with the first reagent after it has found its target and thereby supplying the detectable label. For example, labeled anti-mouse IgG may be provided as a secondary reagent for use with intact 4B5. Labeled avidin may be provided as a secondary reagent when the primary reagent has been conjugated with biotin.

The kits can be employed to test a variety of biological samples, including both liquid samples, cell suspensions and tissue samples. Suitable assays using 4B5 that can be supplied in kit form include those described herein. Each reagent is supplied in a solid form or dissolved/suspended in a liquid buffer suitable for inventory storage, and later for exchange or addition into the reaction medium when the test is performed. Suitable packaging is provided. The kit can optionally provide additional components that are useful in the procedure. These optional components include, but are not limited to, buffers, capture reagents, developing reagents, labels, reacting surfaces, means for detection, control samples, instructions, and interpretive information.

The foregoing description provides, inter alia, detailed methods for preparing 4B5, along with 4B5 encoding polynucleotides, 4B5 polypeptide fragments, and other derivatives. A practitioner of ordinary skill in the art can practice embodiments of this invention by referring to the sequence data for 4B5, which is provided herein. The following examples are provided to illustrate but not limit the claimed invention.

### EXAMPLE 1

### Purification of Recombinant 4B5

4B5 antibodies contained in the hybridoma cell-culture medium or in the lysates of 4B5-expressing cells were purified on a protein G Sepharose column according to standard procedures known in the art (for a reference, see “Methods in Enzymology” (Academic Press, Inc.)). Fractions concentrated with purified antibodies were collected, analyzed by SDS-polyacrylamide gel electrophoresis, and visualized by staining. The results are shown in FIG. 2. A 55-kDa and a 23-kDa protein, consistent with the molecular weight of the heavy chain (55-kDa) and the light chain (23-kDa) of irununoglobulin were detected in samples containing recombinant or hybridoma 4B5. An additional band of approximately 110 kDa, consistent with the molecular weight of a heavy chain/light chain dimer, was also detected in the sample containing recombinant 4B5. This was most likely due to incomplete reduction of the disulfide bonds linking the two immunoglobulin chains.

### EXAMPLE 2

### Antigenic Similarity between Recombinant and Hybridoma 4B5 Determined by ELISA

In order to determine the ability of recombinant 4B5 to bind to antigen 14G2A, ELISA plates were coated with 1 μg/ml of 14G2A or mouse IgG as a negative control. The coated ELISA plates were incubated for 16-18 hours at 2-8° C. The plates were blocked with PBS-3% BSA for 1 hr at room temperature. Then the plates were incubated with either recombinant 4B5 or hybridoma 4B5 in PBS or control human IgG in PBS or culture medium for 2 hrs at room temperature. The plates were washed and incubated with biotinylated anti-human IgG followed by incubation with streptavidin-conjugated alkaline phosphatase for 1 hr. After washing, p-nitrophenyl phosphate substrate was added to each plate and, after incubation, the plates were read at 405 nm in an ELISA plate reader.

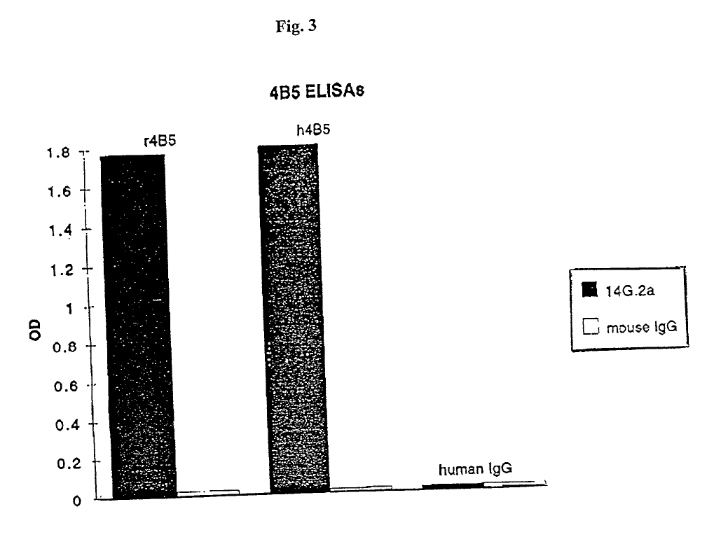

Specific binding of recombinant 4B5 to antigen 14G2A is shown in FIG. 3. These results indicate that recombinant 4B5 has antigenic binding specificity indistinguishable from that of the hybridoma 4B5 antibodies.

### EXAMPLE 3

### Antigenic Specificity of Recombinant and Hybridoma 4B5 Determined by Western Blot Analysis

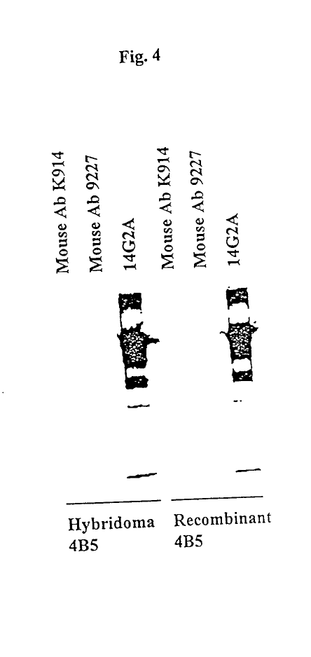

Equal quantities of antigen 14G2A and the control sample, mouse monoclonal antibodies K914 and 9227, were first analyzed by SDS-polyacrylamide gel electrophoresis, and then immunoblotted with either recombinant 4B5 or hybridoma 4B5 under standard conditions (for a reference, see “Methods in Enzymology” (Academic Press, Inc.)). Both recombinant 4B5 and hybridoma 4B5 reacted with antigen 14G2A and not with the mouse monoclonal antibodies. The results are shown in FIG. 4.

Although the foregoing invention has been described in some detail by way of illustration and example for purposes of clarity and understanding, it will be apparent to those skilled in the art that certain changes and modifications may be practiced. Therefore, the description and examples should not be construed as limiting the scope of the invention, which is delineated by the appended claims.

We claim:

1. A substantially isolated polynucleotide sequence that encodes an antigen binding fragment of an antibody, having the amino acid sequences of SEQ ID NOS: 3 and 6.

2. A substantially isolated polynucleotide sequence that encodes at least five consecutive amino acid residues of SEQ ID NOS: 3 or 6.

3. The polynucleotide according to claim 1, wherein the encoding sequence is within SEQ ID NO: 1.

4. The polynucleotide according to claim 3, wherein the encoding sequence is within SEQ ID NO: 4.

5. The polynucleotide according to claim 3, wherein the polynucleotide encodes at least five consecutive amino acid residues of a CDR.

6. An isolated polynucleotide comprising a region of at least 20 consecutive nucleotides that is capable of selectively forming a stable duplex with a polynucleotide consisting of SEQ ID NO: 2 or 5.

7. An isolated polynucleotide comprising a region of at least 20 consecutive nucleotides that is capable of selectively forming a stable duplex with a polynucleotide consisting of SEQ ID NO: 1 or 4.

8. The polynucleotide according to claim 1, wherein the polynucleotide comprises a cloning vector.

9. The polynucleotide according to claim 1, wherein the polynucleotide comprises an expression vector.

10. The expression vector according to claim 9, wherein the expression vector is vaccinia.

11. A host cell comprising a recombinant polynucleotide according to claim 7.

12. A pharmaceutical composition comprising the polynucleotide of claims 1, 2, 3, 4, 5, 6 or 7 and a pharmaceutically acceptable excipient.

13. An immunogenic composition comprising the polynucleotide sequence according to claim 1 and a pharmaceutically acceptable excipient.

14. A composition comprising an antigen binding fragment of an antibody having the amino acid sequence of SEQ ID NO: 3 and a L chain V region having the amino acid sequence of SEQ ID NO: 6.

15. The composition according to claim 14, wherein the antigen binding fragment is selected from the group consisting of whole native antibodies, bispecific antibodies, chimeric antibodies, Fab, F(ab′)2, single chain V region fragments (scFv) and fusion polypeptides, wherein the fusion polypeptide comprises the antigen binding fragment fused to a chemically functional moiety.

16. The composition according to claim 15 wherein the whole native antibody is a 4B5 antibody.

17. The composition according to claim 16, wherein the 4B5 antibody comprises H chains having the amino acid sequence of SEQ ID NO: 3 and a L chain having the amino acid sequence of SEQ ID NO: 6.

18. The composition according to claim 15, wherein the moiety is selected from the group consisting of signal peptides, agents that enhance immunologic reactivity, agents that facilitate coupling to a solid support, vaccine carriers, bioresponse modifiers, toxins, detectable labels, paramagnetic labels, and drugs.

19. The composition according to claim 18, wherein the agent that enhances immunologic reactivity is a bacterial super antigen.

20. The method according to claim 18, wherein the agent that facilitates coupling to a solid support is selected from the group consisting of biotin and avidin.

21. The composition according to claim 18, wherein the immunogen carrier is selected from the group consisting of any physiologically acceptable buffer.

22. The composition according to claim 18, wherein the bioresponse modifier is a cytokine.

23. The composition according to claim 22, wherein the cytokine is selected from the group consisting of tumor necrosis factor, interleukin-2, interleukin-4, interleukin- 12, granulocyte macrophage colony stimulating factor and γ-interferons.

24. The composition according to claim 18, wherein the detectable label is selected from the group consisting of radioisotopes, fluorescent compounds, colloidal metals, chemiluminescent compounds, bioluminescent compounds, enzymes, substrates, cofactors and inhibitors.

25. A polypeptide comprising at least five consecutive amino acid residues of SEQ ID NOS: 3 or 6.

26. The polypeptide according to claim 25, wherein the five consecutive amino acid residues are from a CDR.

27. The polypeptide according to claim 25, further comprising a heterologous immunoglobulin C region.

28. A polymeric peptide comprising a plurality of the peptide according to claim 25.

29. The composition according to claim 25, further comprising a pharmaceutically acceptable excipient.

30. The composition according to claim 29, wherein the excipient is a liposome preparation.

31. An immunogenic composition comprising the composition according to claim 25, further comprising a pharmaceutically acceptable excipient and an amount of an adjuvant effective to enhance the immune response.

32. A method of treating a patient with a neoplasia comprising administering to the patient an effective amount of the composition according to claim 25.

33. The method according to claim 32, wherein the individual has a clinically detectable tumor.

34. The method according to claim 32, which is a method for palliating the neoplasia.

35. The method according to claim 32, wherein a tumor that was previously detected in the individual has been treated and is clinically undetectable at the time of the administering of the antigen binding fragment.

36. The method according to claim 32, which is a method of reducing the risk of recurrence of a clinically detectable tumor.

37. The method according to claim 32, wherein administration of the antigen binding fragment is by parenteral administration selected from the group consisting of subcutaneous, intramuscular, intraperitoneai, intmcavity, intrathecal, transdermal, or intravenous injection.

38. The method according to claim 32, wherein the antigen binding fragment is labeled with a therapeutic moiety.

39. The method according to claim 38, wherein the therapeutic moiety is selected from the group consisting of radioisotopes, antineoplastic agents, immunomodulators, biological response modifiers, lectins and toxins.

40. A composition comprising substantially purified recombinant 4B5-antigen, comprising a H chain V region having the amino acid sequence of SEQ ID NO: 3 and a L chain V region having the amino acid sequence of SEQ ID NO: 6.

41. The composition according to claim 40, wherein the 4B5-antigen is present in an immunogenic amount and further wherein the composition includes an amount of adjuvant effective to enhance an immune response to the GD2.

42. A method for detecting antibodies specific for GD2 in a sample, comprising the steps of:
a) contacting the sample with the composition according to claim 14 under conditions that permit the formation of a stable antibody-antigen complex; and
b) detecting any stable complex formed in step a).

43. A kit for detecting or quantification of polynucleotide comprising a polynucleotide encoding 4B5 or a fragment thereof in a biological sample, said kit comprising the polynucleotide of claim 1 in suitable packaging.

44. A kit for detecting or quantification of 4B5-antigen in a biological sample, said kit comprising the antibody in claim 14 in suitable packaging.
